(19) United States
(12) Patent Application Publication
(10) Pub. No.: US 2020/0085734 A1
(43) Pub. Date: Mar. 19, 2020

(54) PHARMACEUTICAL DOSAGE FORMS CONTAINING TASK-I AND TASK-3 CHANNEL INHIBITORS, AND THE USE OF SAME IN BREATHING DISORDER THERAPY

(71) Applicant: Bayer Pharma Aktiengesellschaft, Berlin (DE)

(72) Inventors: Johanna ANLAHR, Dortmund (DE); Moritz BECK-BROICHSITTER, Darmstadt (DE); Janine NICOLAI, Essen (DE); Martina DELBECK, Heiligenhaus (DE); Michael HAHN, Langenfeld (DE); Udo ALBUS, Florstadt (DE); Doris GEHRING, Kelkheim (DE); Björn ROSENSTEIN, Bad Soden-Salmünster (DE)

(73) Assignee: Bayer Pharma Aktiengesellschaft, Berlin (DE)

(21) Appl. No.: 16/471,263

(22) PCT Filed: Dec. 13, 2017
(86) PCT No.: PCT/EP2017/082545

§ 371 Date: Jun. 19, 2019

(30) Foreign Application Priority Data
Dec. 21, 2016 (EP) 16205686.5
Feb. 24, 2017 (EP) 17157800.8

(51) Int. Cl. A61K 9/00; A61K 47/10; A61K 31/496; A61K 47/26

(52) U.S. Cl. CPC A61K 9/0043; A61K 47/26; A61K 31/496; A61K 47/10

(57) ABSTRACT

The invention relates to new pharmaceutical dosage forms containing potent and selective TASK-1 and/or TASK-3 channel inhibitors, and the use of same to treat and/or prevent breathing disorders including sleep-related breathing disorders such as obstructive and central sleep apnoea and snoring.

The present application relates to novel dosage administration forms comprising potent and selective inhibitors of TASK-1 and/or TASK-3 channels and use thereof for the treatment and/or prevention of respiratory disorders, including sleep-related respiratory disorders such as obstructive and central sleep apnoeas and snoring.

Potassium channels are virtually ubiquitous membrane proteins which are involved in a large number of different physiological processes. This also includes the regulation of the membrane potential and the electric excitability of neurons and muscle cells. Potassium channels are divided into three major groups which differ in the number of transmembrane domains (2, 4 or 6). The group of potassium channels where two pore-forming domains are flanked by four transmembrane domains is referred to as K2P channels (Two-pore domain K⁺). Functionally, the K2P channels mediate, substantially time- and voltage-independently, K⁺ background currents, and their contribution to the maintenance of the resting membrane potential is crucial. The family of the K2P channels includes 15 members which are divided into six subfamilies, based on similarities in sequence, structure and function: TWIK (tandem pore domain halothane inhibited K⁺ channel), TREK (TWIK-related K⁺ channel), TASK (TWIK-related acid-sensitive K⁺ channel), TALK (TWIK-related alkaline pH activated K⁺ channel), THIK (tandem pore domain halothane inhibited K⁺ channel) and TRESK (TWIK-related spinal cord K⁺ channel).

Of particular interest are TASK-1 (KCNK3 or K2P3.1) and TASK-3 (KCNK9 or K2P9.1) of the TASK (TWIK-related acid-sensitive K⁺ channel) subfamily. Functionally, these channels are characterized in that, during maintenance of voltage-independent kinetics, they have “leak” or “background” currents flowing through them, and they respond to numerous physiological and pathological influences by increasing or decreasing their activity. Characteristic of TASK channels is the sensitive reaction to a change in extracellular pH: the channels are inhibited at acidic pH and activated at alkaline pH.

TASK-1 and TASK-3 channels play a role in respiratory regulation. Both channels are expressed in the respiratory neurons of the respiratory centre in the brain stem, inter alia in neurons which generate the respiratory rhythm (ventral respiratory group with pre-Bötzinger complex), and in the noradrenergic Locus caeruleus, and also in serotonergic neurons of the raphe nuclei. Owing to the pH dependency, here the TASK channels have the function of a sensor which translates changes in extracellular pH into corresponding cellular signals [Bayliss et al., Pflugers Arch. 467, 917-929 (2015)]. TASK-1 and TASK-3 are also expressed in the Glomus caroticum, a paraganglion, which measures the pH and the O₂ and CO₂ content of the blood and transmits signals to the respiratory centre in the brain stem to regulate respiration. It was shown that TASK-1 knock-out mice have a reduced ventilatory response (increase of respiratory rate and tidal volume) to hypoxia and normoxic hypercapnia [Trapp et al., J. Neurosci. 28, 8844-8850 (2008)]. Furthermore, TASK-1 and TASK-3 channels were demonstrated in motoneurons of the Nervus hypoglossus, the XIIth cranial nerve, which has an important role in keeping the upper airways open [Berg et al., J. Neurosci. 24, 6693-6702 (2004)].

In a sleep apnoea model in the anaesthetized pig, nasal administration of a potassium channel blocker which blocks the TASK-1 channel in the nanomolar range led to inhibition of collapsibility of the pharyngeal airway musculature and sensitization of the negative pressure reflex of the upper airways. It is assumed that nasal administration of the potassium channel blocker depolarizes mechanoreceptors in the upper airways and, via activation of the negative pressure reflex, leads to increased activity of the musculature of the upper airways, thus stabilizing the upper airways and preventing collapse. By virtue of this stabilization of the upper airways, the TASK channel blockade may be of great importance for obstructive sleep apnoea and also for snoring [Wirth et al., Sleep 36, 699-708 (2013); Kiper et al., Pflugers Arch. 467, 1081-1090 (2015)].

Obstructive sleep apnoea (OSA) is a sleep-related respiratory disorder which is characterized by repeat episodes of obstruction of the upper airways. When breathing in, the patency of the upper airways is ensured by the interaction of two opposite forces. The dilative effects of the musculature of the upper airways counteract the negative intraluminal pressure, which constricts the lumen. The active contraction of the diaphragm and the other auxiliary respiratory muscles generates a negative pressure in the airways, thus constituting the driving force for breathing. The stability of the upper airways is substantially determined by the coordination and contraction property of the dilating muscles of the upper airways.

The Musculus genioglossus plays a decisive role in the pathogenesis of OSA. The activity of the Musculus genioglossus increases with decreasing pressure in the pharynx in the sense of a dilative compensation mechanism. Innervated by the Nervus hypoglossus, it drives the tongue forward and downward, thus widening the pharyngeal airway [Verse et al., Somnologie 3, 14-20 (1999)]. Tensioning of the dilating muscles of the upper airways is modulated inter alia via mechanoreceptors/stretch receptors in the nasal cavity/pharynx [Bouillette et al., J. Appl. Physiol. Respir. Environ. Exerc. Physiol. 46, 772-779 (1979)]. In sleeping patients suffering from serious sleep apnoea, under local anaesthesia of the upper airway an additional reduction of the activity of the Musculus genioglossus can be observed [Berry et al., Am. J. Respir. Crit. Care Med. 156, 127-132 (1997)]. Patients suffering from OSA have high mortality and morbidity as a result of cardiovascular disorders such as hypertension, myocardial infarction and stroke [Vrints et al., Acta Clin. Belg. 68, 169-178 (2013)].

In the case of central sleep apnoea, owing to impaired brain function and impaired respiratory regulation there are episodic inhibitions of the respiratory drive. Central respiratory disorders result in mechanical respiratory arrests, i.e. during these episodes there is no breathing activity; temporarily, all respiratory muscles including the diaphragm are at rest. In the case of central sleep apnoea, there is no obstruction of the upper airways.

In the case of primary snoring, there is likewise no obstruction of the upper airways. However, owing to the constriction of the upper airways, the flow rate of the air that is inhaled and exhaled increases. This, combined with the relaxed musculature, causes the soft tissues of the oral cavity and the pharynx to flutter in the stream of air. This gentle vibration then generates the typical snoring noises.

Obstructive snoring (upper airway resistance syndrome, heavy snoring, hypopnoea syndrome) is caused by repeat partial obstruction of the upper airways during sleep. This results in an increased airway resistance and thus in an increase in work of breathing with considerable fluctuations in intrathoracic pressure. During inspiration, the development of negative intrathoracic pressure may reach values similar to those that are encountered as a result of complete airway obstruction during OSA. The pathophysiological consequences for heart, circulation and sleep quality correspond to those of obstructive sleep apnoea. As in OSA, the pathogenesis can be assumed to be an impaired reflex mechanism of the pharynx-dilating muscles during inspiration when sleeping. Frequently, obstructive snoring is the preliminary stage of OSA [Hollandt et al., HNO 48, 628-634 (2000)].

The currently available therapeutic possibilities for snoring and OSA are limited. Mixtures of surface-active substances have been known since the 1980s which are intended to reduce the resistance of the upper airways and snoring [Widdicombe and Davies, Eur Resp J 1, 785-791 (1988)]. These mixtures comprise NaCl, glycerol, polysorbate 80 and benzalkonium chloride. From experiments in dogs, to which these mixtures were administered by injection into the pharynx, it was concluded that these mixtures reduce the resistance of the upper airways, increase the activity of the Musculus genioglossus when breathing in and breathing out and reduce snoring noises. OSA is not mentioned in the article by Widdicombe and it has also not been shown in this model that a collapse of the upper airways, which leads to apnoea, could be prevented. The model of Widdicombe and Davies is therefore not predictive for OSA.

A composition consisting of: 0.26% glycerol, 0.2% polysorbate 80, 0.9% sodium chloride and 0.15% potassium sorbate (without benzalkonium chloride) is on the market as Asonor® as a therapy for snoring. In a study at University State Hospital in Copenhagen, the efficacy of nasal administration of Asonor® with respect to improving snoring was investigated in comparison with “Asonor®” without polysorbate 80. Both Asonor® and “Asonor®” without polysorbate 80 effected significant improvement of snoring [Report from the Department of Neurology, University State Hospital, Copenhagen, Denmark. The effect of nasal application of Asonor® and Poly glycoside 80 on snoring and sleep apnoea, 1989, http://www.chrapat.sk/img/klinicka-dokumentacia.pdf].

EP 2595685 B1 (U.S. Pat. No. 9,132,243 B1) claims a pharmaceutical product comprising a container which comprises a liquid anti-snoring substance, wherein the container comprises a liquid outlet section which is configured to deliver the liquid anti-snoring substance directly into the nasal passage in the form of a jet stream. The liquid anti-snoring substance is an anti-snoring solution comprising sodium chloride, glycerol, polysorbate and sodium edetate and optionally potassium sorbate as preservative. A therapy for apnoea or OSA is not disclosed in the original filed application documents of EP 2595685 B1 and U.S. Pat. No. 9,132,243 B1. EP 2595685 B1 claims the anti-snoring substance described for use in the treatment of snoring and respiratory arrest (apnoea).

No pharmacological therapy is currently available for therapy of OSA. Operations and oral devices are of only limited efficacy. The treatment standard is therapy with the continuous positive airway pressure (CPAP) system. The compliance rate of this therapy, due to the discomfort, is only 50-70% and the system is used on average not more than 4 hours per night.

Novel substances, which act as potent and selective inhibitors of TASK-1 and/or TASK-3 channels and are suitable as such in particular for the treatment and/or prevention of respiratory disorders, including sleep-related respiratory disorders such as obstructive and central sleep apnoeas and snoring and also other disorders, are known from PCT/EP2016/079973 and PCT/EP2016/079544 (unpublished).

The duration of action of the potent and selective inhibitors of TASK-1 and/or TASK-3 channels disclosed in EP 15199270.8 and EP 15199268.2 on nasal administration is not always sufficient, which makes redosing during the night and therefore interruption of the night's rest or sleep necessary.

The object of the present invention, therefore, is to provide an effective pharmacological therapy for the treatment and/or prevention of respiratory disorders, including sleep-related respiratory disorders such as obstructive and central sleep apnoeas and snoring, which represents an alternative to the treatment with the CPAP system.

A further object of the present invention is to increase the rate of compliance by the patients of a treatment and/or prevention of respiratory disorders, including sleep-related respiratory disorders such as obstructive and central sleep apnoeas and snoring, compared to the current therapy standard (therapy of OSA: CPAP system). For this purpose, this alternative therapy should be simple and comfortable to use and not disturb the person sleeping. In addition, this alternative therapy should enable an undisturbed night's rest without repeat medication with a once daily dose prior to going to sleep.

A further object of the present invention, therefore, is to provide the pharmacologically effective substances for the treatment and/or prevention of respiratory disorders, including sleep-related respiratory disorders such as obstructive and central sleep apnoeas and snoring, in an administration form which is suitable for once daily nasal or pharyngeal administration prior to going to sleep. In particular, it is an object of the present invention to provide a pharmacologically effective therapy for the treatment and/or prevention of respiratory disorders, including sleep-related respiratory disorders such as obstructive and central sleep apnoeas and snoring, which has a duration of action of at least 4 hours.

Extending the duration of action of nasally administered active ingredients is difficult. Due to physiological conditions, the residence time of active ingredients, particles, capsules and the like in the epithelial cells is short. The epithelium consists in part of cilial cells which have hair-like structures, the cilia. These are covered by a mucous layer which is transported away towards the throat by a coordinated movement of the cilia. Foreign particles and microorganisms remain adhering to the mucous layer after nasal uptake and are transported towards the throat and oesophagus by mucociliary clearance together with the mucous. Mucociliary clearance therefore counteracts the nasal absorption of active ingredients and is in particular a challenge for achieving a prolonged effect. The mucous flow rate is about 5 mm per minute and therefore it is renewed every 15-20 min. Clearance half-lives of 15 min were therefore also determined for nasally administered solutions and powders [Illum et al., Int J Pharm. 39, 189-199 (1987)], and therefore active ingredients in principle remain only briefly on the mucosa in order to achieve an effect.

A method for achieving a prolongation of effect after nasal administration is to prolong the contact time between active ingredient and the absorption site, the epithelial cells, in the nose. The absorption of medicaments in the nose is increased by a prolonged contact time. The active ingredient uptake can occur over a longer period so that firstly a prolonged effect and duration of action may be achieved and secondly the total amount of medicament absorbed may be increased. Methods to increase the contact time between the active ingredient and the epithelial cells are, inter alia, increasing the viscosity, the use of bioadhesive polymers or the use of microparticles.

Pennington et al. could already show in 1988 that the clearance rate is reduced by increasing the viscosity of nasally administered solutions with hydroxypropylmethylcellulose [Pennington et al., Int J Pharm. 43, 221-224 (1988)]. With increasing polymer proportion and thus increasing viscosity, the half-life increased from 1 hour to 2.2 hours. Compared with the half-lives of solutions of 15 min observed by Illum et al. [Illum et al., Int J Pharm. 39, 189-199 (1987)], increasing the viscosity thus led to a distinct prolongation of the half-life. Viscous solutions and semi-solid systems such as gels, creams and ointments can however be more difficult to apply than low-viscosity formulations. Atomization via a spray is no longer possible and a precise dosage with the aid of applicators in the case of semi-solid systems is difficult. In addition, nasally applied semi-solid systems may lead to a blockage which may disrupt nasal breathing. In addition to the administration of higher viscosity solutions and ready-to-apply gels, the administration of in situ gels is also conceivable [Majithiya et al., AAPS PharmSciTech 7 (3), Article 67 (2006)]. Here, the gelation is first triggered within the nose, for example by a temperature change, a change of pH or by the presence of ions. In this way, a low-viscosity solution can be applied and the viscous formulation is available after gelation at the site of deposition, the nasal mucosa, with positive effects therefrom. Metering systems can thus be used for the administration which enable a precise and simple administration. However, they are complex and elaborate dosage forms since the gel formation has to be precisely coordinated. If the gelation is caused by a temperature change for example, it must be ensured that the gelation is only triggered at physiological temperatures and is still suppressed on storage. Therefore, particular requirements on storage and handling are applied on the one hand in order to prevent premature gelation while on the other hand the development and manufacturing complexity of such a sensitive system is very high.

Starch and chitosan are frequently used as bioadhesive polymers [Illum et al., J Controlled Release 87, 187-198 (2003)]. Chitosan is a bioadhesive polysaccharide and can interact markedly with the epithelial cells and the mucous layer. A longer contact time is thereby produced which allows the active ingredient transport through the membrane. Chitosan is widely used in the literature, however it is used predominantly in in vitro experiments. Chitosan is currently not approved for nasal administration (FDA Drug Databases, Inactive Ingredient Search for Approved Drug Products) and the potential long-term toxicity for chronic nasal administration is not fully investigated.

A further possibility to prolong the effect after nasal active ingredient administration is the encapsulation of the active ingredient in polymeric microparticles [Cerchiara et al., Eur J Pharm Biopharm. 61, 195-200 (2005)]. For this purpose, the active ingredient is embedded in a suitable polymer which has a low solubility in water, or a polymer combination which additionally enables adhesion of the active ingredient-laden microparticles to the nasal mucosa. After introduction of this dosage form into the nose, the active ingredient is released in a time-delayed manner from the microparticles by diffusion and/or polymer degradation/erosion, depending on the property of the polymer used, which results in a prolonged duration of action of the active ingredient at the site of action. If the polymer combination used, from which the microparticles are composed, additionally has the property of adhering to the nasal mucosa, a prolonged residence time and hence duration of action of the nasally introduced medication is to be expected. Just the combination of microparticles and bioadhesive polymers therefore represents a much described approach for prolonging the duration of action on nasal administration, since two priniciples here—the delayed release and the increased contact time—are combined. In this case, the microparticles can be prepared directly from a bioadhesive polymer [Illum et al., Int J Pharm. 39, 189-199 (1987)] or other polymers such as poly(lactide-co-glycolide) (PLGA) can be used to produce the microparticles which are then coated with the bioadhesive polymer in a further step [Pawar et al., Am Assoc Pharmac Sci J 12, 130-137 (2010)].

In addition to the use of the microparticles described above, the active ingredient release can also be prolonged by the use of suspended instead of dissolved active ingredient. For this purpose, the active ingredient used is micronized for example (comminution to active ingredient microparticles) and incorporated in a liquid phase (suspended). After administration in the nose, the active ingredient particles dissolve in a delayed manner at the site of action. Only the dissolved active ingredient can be absorbed through the nasal mucosa and then be effective. The dissolution kinetics, which determines the prolongation of the active effect, depends on, inter alia, the physicochemical properties (e.g. solubility, particle size) of the active ingredient used. By administering crystal suspensions of glucocorticoids, a local prolongation of effect can be achieved for example [Rygg et al., Pharm Res. 33, 909-921(2016)].

The processing of active ingredients in crystal suspensions and encapsulating active ingredients in polymeric microparticles with the aim of prolonging the effect after nasal administration is linked to numerous disadvantages.

Firstly, the production of such dosage forms is technically many times more complex in comparison to, for example, active ingredient solutions. For instance, the production of crystal suspensions and polymeric microparticles requires numerous successive process steps which significantly influence the quality of the finished dosage form. The functionality of these complex dosage forms can be unfavourably influenced owing to lack of storage stability. For instance, crystal suspensions exhibit, for example, particle sedimentation (incl. sediment formation) and/or changes to the primary particle size during storage, which leads to inhomogeneity within the dosage form and therefore dosing errors.

Secondly, the production of crystal suspensions and polymeric microparticles requires the use of numerous stabilizers and polymeric matrix formers which can result in local intolerances/irritations following nasal administration. For example, it is known that numerous stabilizers can lead to undesirable influence on the cilia motility, cell lysis and inactivation of enzymes [Schinichiro et al., Int J Pharm. 9, 173-184 (1981)]. During the hydrolytic degradation of polymers such as bioresorbable polyesters (e.g. PLGA), which are frequently used as matrix formers for microparticles, release of degradation products (e.g. lactic acid and glycolic acid) occurs, which can significantly lower the local pH, whereby local irritation may occur. Local irritations can also be triggered by the particles themselves.

Moreover, just the use of particulate systems such as crystal suspensions and polymeric microparticles, which are accompanied by a delayed release and dissolution of the active ingredient, can lead to a non-reproducible proportion of the dose being transported out and swallowed as undissolved particles prior to absorption due to mucociliary clearance. Swallowing of active ingredient can in turn lead to a large variability in exposure [Malinovsky et al., Br J Anaesthesia 77, 203-207 (1996)].

Furthermore, the use of crystal suspensions and polymeric microparticles is linked to complex instructions for use, which may lead to application errors, which in turn jeopardize the therapeutic response desired.

Disadvantages of the approaches described for prolonging the effect of nasally administered active ingredients, such as viscous systems, crystal suspensions and microparticles, are accordingly the high expenditure in the production, the complexity of these dosage forms, the risk of high variability in exposure and not least the inadequate safety of the auxiliaries used (e.g. polymers) for nasal administration.

It has been shown in the present invention, surprisingly, that nasal administration of a formulation comprising a therapeutically effective amount of at least one inhibitor of the TASK-1 and/or TASK-3 channel, or a hydrate, solvate, polymorph or metabolite thereof or a pharmaceutically acceptable salt thereof in >2.5% to 100% w/v polyethylene glycol significantly prolongs the duration of action of the inhibitor of the TASK-1 and/or TASK-3 channel, or a hydrate, solvate, polymorph or metabolite thereof or a pharmaceutically acceptable salt thereof, depending on the dose.

The present invention provides stable pharmaceutical formulations for nasal or pharyngeal administration comprising a therapeutically effective amount of at least one inhibitor of the TASK-1 and/or TASK-3 channel or a hydrate, solvate, polymorph or metabolite thereof or a pharmaceutically acceptable salt thereof in >2.5% to 100% w/v polyethylene glycol and optionally at least one auxiliary, wherein the formulation has a pH of 4 to 8.

A nasal or pharyngeal administration of a therapeutically effective amount of at least one inhibitor of the TASK-1 and/or TASK-3 channel or a hydrate, solvate, polymorph or metabolite thereof or a pharmaceutically acceptable salt thereof in a formulation comprising a pH regulator and a solubilizer without addition of polyethylene glycol did not lead to prolonging the duration of action even on increasing the dose of the inhibitor of the TASK-1 and/or TASK-3 channel.

Surprisingly, formulations comprising a therapeutically effective amount of at least one inhibitor of the TASK-1 and/or TASK-3 channel or a hydrate, solvate, polymorph or metabolite thereof or a pharmaceutically acceptable salt thereof and comprising 20% w/v of propylene glycol (instead of polyethylene glycol), and a pH regulator and a solubilizer did not show any prolongation of the duration of action of the inhibitor of the TASK-1 and/or TASK-3 channel.

Also formulations comprising a therapeutically effective amount of at least one inhibitor of the TASK-1 and/or TASK-3 channel or a hydrate, solvate, polymorph or metabolite thereof or a pharmaceutically acceptable salt thereof and 1.25% w/v of the viscosity-enhancing substance Na carboxymethyl cellulose (Na-CMC) (instead of PEG), and a pH regulator and a solubilizer did not show any prolongation of the duration of action of the inhibitor of the TASK-1 and/or TASK-3 channel, although the viscosity of this formulation with 1.25% w/v Na-CMC is comparable with the viscosity of a formulation according to the invention comprising 20% w/v PEG 400. This indicates that an increase in viscosity due to addition of PEG cannot be the decisive reason for the prolongation of the duration of action observed with the formulations according to the invention.

A composition comprising a polysorbate 80 and at least 5% w/v PEG 400 in phosphate buffer, pH 7,without an inhibitor of the TASK-1 and/or TASK-3 channel also showed no effect in the present invention.

A person skilled in the art has no starting point with which to replace the physical therapy of OSA by CPAP, since a pharmacological alternative is described for the first time in the unpublished PCT/EP2016/079973. There are also currently no, or only very limited, pharmacological therapies for snoring, and therefore a person skilled in the art would even here have had no starting point to get to the present invention. Even if the TASK-1 and/or TASK-3 inhibitors described in PCT/EP2016/079973 would have been known, the person skilled in the art would have had no reason to assume that the very simply manageable solution outlined for prolonging the duration of action of the inhibitor of the TASK-1 and/or TASK-3 channel is successful.

There is no indication in the prior art that prolongation of the effect of inhibitors of the TASK-1 and/or TASK-3 channel by several hours with regard to OSA can be achieved by the use of the standard formulation auxiliary polyethylene glycol but not with propylene glycol. There is also no indication in the prior art that prolongation of effect by several hours can be achieved without the use of complex approaches such as microparticles, crystal suspensions or bioadhesive systems described in the prior art for prolonging the effect of nasally administered active ingredients.

In addition, there is no indication in the prior art that the prolongation of effect with the aid of the formulations according to the invention can only be achieved in a specific concentration range of the formulation constituent polyethylene glycol. An indication of suitable concentration ranges of the formulation constituents is also not found in the prior art.

In the context of the present invention, the stable pharmaceutical formulation is administered by the nasal or pharyngeal route.

In the context of the present invention, the terms “nasal” and “intranasal” are used synonymously.

In the context of the present invention, stable pharmaceutical formulations which are suitable for nasal administration are formulations in liquid, semi-solid or solid form, for example nasal drops, nasal solutions, nasal gels, nasal ointments, nasal creams or pulverulent dosage forms.

In the context of the present invention, nasal administration can be effected by means of, for example, nasal spray, dropping pipette, squeeze bottle, COMOD® system, liquid atomizers (e.g. piezoelectric nebulizers, nozzle or ultrasound aerosol generators, soft mist inhalers) or metered-dose aerosols, or nasal applicators for semi-solid formulations (syringe tubes, spatula) and/or solid formulations (powder). According to one embodiment of the present invention, the administration is effected by nasal spray.

In the context of the present invention, stable pharmaceutical formulations which are suitable for pharyngeal administration are formulations in liquid, semi-solid or solid form, for example solutions, gels or powders.

In the context of the present invention, pharyngeal administration can be effected by means of inhalation using liquid atomizers (e.g. piezoelectric nebulizers, nozzle or ultrasound aerosol generators, pump sprays) or metered-dose aerosols, or by means of local administration using a bronchoscope (instillation), a dropping pipette, squeeze bottle or similar.

In the context of the present invention, the therapeutic effect is defined as a reduction of the apnoea-hypopnoea index (AHI) of a patient with sleep-related respiratory disorders such as obstructive and central sleep apnoeas and snoring after nasal or pharyngeal administration of a formulation according to the invention comprising a therapeutically effective amount of at least one inhibitor of the TASK-1 and/or TASK-3 channel or a hydrate, solvate, polymorph or metabolite thereof or a pharmaceutically acceptable salt thereof.

According to one embodiment of the present invention, the therapeutic effect is defined as a reduction by at least 20% of the apnoea-hypopnoea index (AHI) of a patient with sleep-related respiratory disorders such as obstructive and central sleep apnoeas and snoring after nasal or pharyngeal administration of a formulation according to the invention comprising a therapeutically effective amount of at least one inhibitor of the TASK-1 and/or TASK-3 channel or a hydrate, solvate, polymorph or metabolite thereof or a pharmaceutically acceptable salt thereof.

According to one embodiment of the present invention, the therapeutic effect is defined as a reduction by at least 20%, at least 25%, at least 30%, at least 35%, at least 40%, at least 45%, at least 50%, at least 55%, at least 60%, at least 65%, at least 70%, at least 75% or at least 80% of the apnoea-hypopnoea index (AHI) of a patient with sleep-related respiratory disorders such as obstructive and central sleep apnoeas and snoring after nasal or pharyngeal administration of a formulation according to the invention comprising a therapeutically effective amount of at least one inhibitor of the TASK-1 and/or TASK-3 channel or a hydrate, solvate, polymorph or metabolite thereof or a pharmaceutically acceptable salt thereof.

In the context of the present invention, the duration of action is defined as the period in which the apnoea-hypopnoea index (AHI) of said patient is reduced after nasal or pharyngeal administration of a formulation according to the invention comprising a therapeutically effective amount of at least one inhibitor of the TASK-1 and/or TASK-3 channel or a hydrate, solvate, polymorph or metabolite thereof or a pharmaceutically acceptable salt thereof, to a patient with sleep-related respiratory disorders such as obstructive and central sleep apnoeas and snoring.

According to one embodiment of the present invention, the duration of action is defined as the period in which the apnoea-hypopnoea index (AHI) of said patient is reduced by at least 20% after nasal or pharyngeal administration of a formulation according to the invention comprising a therapeutically effective amount of at least one inhibitor of the TASK-1 and/or TASK-3 channel or a hydrate, solvate, polymorph or metabolite thereof or a pharmaceutically acceptable salt thereof, to a patient with sleep-related respiratory disorders such as obstructive and central sleep apnoeas and snoring.

According to one embodiment of the present invention, the duration of action is defined as the period in which the apnoea-hypopnoea index (AHI) of said patient is reduced by at least 20%, at least 25%, at least 30%, at least 35%, at least 40%, at least 45%, at least 50%, at least 55%, at least 60%, at least 65%, at least 70%, at least 75% or at least 80% after nasal or pharyngeal administration of a formulation according to the invention comprising a therapeutically effective amount of at least one inhibitor of the TASK-1 and/or TASK-3 channel or a hydrate, solvate, polymorph or metabolite thereof or a pharmaceutically acceptable salt thereof, to a patient with sleep-related respiratory disorders such as obstructive and central sleep apnoeas and snoring.

In the context of the present invention, the duration of action is at least 3 hours or at least 3.5 hours or at least 4 hours or at least 4.5 hours or at least 5 hours or at least 5.5 hours or at least 6 hours or at least 6.5 hours or at least 7 hours or at least 7.5 hours or at least 8 hours. According to one embodiment of the present invention, the duration of action is at least 3 hours. According to one embodiment of the present invention, the duration of action is at least 4 hours. According to one embodiment of the present invention, the duration of action is at least 5 hours.

In the context of the present invention, a therapeutically effective amount of at least one inhibitor of the TASK-1 and/or TASK-3 channel, or a hydrate, solvate, polymorph or metabolite thereof or a pharmaceutically acceptable salt thereof, is defined as the amount of at least one inhibitor of the TASK-1 and/or TASK-3 channel, or a hydrate, solvate, polymorph or metabolite thereof or a pharmaceutically acceptable salt thereof, which on nasal or pharyngeal administration shows a duration of action of at least 3 hours or at least 3.5 hours or at least 4 hours or at least 4.5 hours or at least 5 hours or at least 5.5 hours or at least 6 hours or at least 6.5 hours or at least 7 hours or at least 7.5 hours or at least 8 hours.

In the context of the present invention, a therapeutically effective amount of at least one inhibitor of the TASK-1 and/or TASK-3 channel, or a hydrate, solvate, polymorph or metabolite thereof or a pharmaceutically acceptable salt thereof, is defined as the amount of at least one inhibitor of the TASK-1 and/or TASK-3 channel, or a hydrate, solvate, polymorph or metabolite thereof or a pharmaceutically acceptable salt thereof, which on nasal or pharyngeal administration shows a duration of action of at least 3 hours.

In the context of the present invention, a therapeutically effective amount of at least one inhibitor of the TASK-1 and/or TASK-3 channel, or a hydrate, solvate, polymorph or metabolite thereof or a pharmaceutically acceptable salt thereof, is defined as the amount of at least one inhibitor of the TASK-1 and/or TASK-3 channel, or a hydrate, solvate, polymorph or metabolite thereof or a pharmaceutically acceptable salt thereof, which on nasal or pharyngeal administration shows a duration of action of at least 4 hours.

In the context of the present invention, a therapeutically effective amount of at least one inhibitor of the TASK-1 and/or TASK-3 channel, or a hydrate, solvate, polymorph or metabolite thereof or a pharmaceutically acceptable salt thereof, is defined as the amount of at least one inhibitor of the TASK-1 and/or TASK-3 channel, or a hydrate, solvate, polymorph or metabolite thereof or a pharmaceutically acceptable salt thereof, which on nasal or pharyngeal administration shows a duration of action of at least 5 hours.

In the context of the present invention, auxiliaries are substances which, in the stable pharmaceutical formulation serve the purpose, for example, of adjusting or stabilizing the pH, of increasing the solubility of the active ingredient, of microbiologically and physically stabilizing the preparation, of modifying the viscosity of the formulation or improving the taste or appearance.

Examples of auxiliaries in the context of the present invention are pH regulators, solubilizers, antioxidants, stabilizers, thickeners, preservatives, substances for adjusting tonicity, aromas, fragrances or dyes.

The present invention also provides stable pharmaceutical formulations according to the invention for nasal or pharyngeal administration, wherein the optional at least one auxiliary is selected from the group consisting of at least one pH regulator, at least one solubilizer, at least one antioxidant, at least one stabilizer, at least one thickener, at least one preservative, at least one substance for adjusting tonicity, at least one aroma, at least one fragrance and at least one dye.

In the context of the present invention, pH regulators are, for example, buffers such as citric acid and salts thereof, acetic acid and salts thereof and phosphoric acid and salts thereof, or inorganic acids such as hydrochloric acid, boric acid, carboxylic acids, dicarboxylic acids, amino acids or organic acids such as monocarboxylic acids such as oxocarboxylic acids or polycarboxylic acids, or bases such as sodium hydroxide, potassium hydroxide, sodium carbonate, sodium hydrogencarbonate.

The present invention also provides stable pharmaceutical formulations according to the invention for nasal or pharyngeal administration, wherein the optional at least one pH regulator is selected from the group consisting of citric acid and salts thereof, acetic acid and salts thereof, phosphoric acid and salts thereof, hydrochloric acid, boric acid, carboxylic acids, dicarboxylic acids, amino acids, oxocarboxylic acids, polycarboxylic acids, sodium hydroxide, potassium hydroxide, sodium carbonate and sodium hydrogencarbonate.

According to one embodiment of the invention, the pH regulator is a phosphate buffer. According to one embodiment of the invention, the pH regulator is a phosphate buffer which buffers the solution in the context of the present invention to a pH between 4 and 8. The preferred pH range is between 7 and 8. According to one embodiment, the pH of the formulations according to the invention is 7.

In the context of the present invention, solubilizers are, for example, chelating agents (for example cyclodextrins and sodium EDTA (sodium ethylenediaminetetraacetate)), cosolvents (for example ethanol, propylene glycol, dimethylacetamide), and surfactants. The group of surfactants includes, for example, fatty alcohols (for example cetyl alcohol), phospholipids (for example lecithin), sterols (for example cholesterol), bile acid salts, saponins, glycerol fatty acid esters (for example glycerol monostearate), polyoxyethylene fatty acid esters (for example polyoxyethylene stearate), polyoxyethylene sorbitan fatty acid esters (such as Tween®, for example polysorbate 20 (polyoxyethylene (20) sorbitan monolaurate), polysorbate 21 (polyoxyethylene (4) sorbitan monolaurate), polysorbate 40 (polyoxyethylene (20) sorbitan monopalmitate), polysorbate 60 (polyoxyethylene (20) sorbitan monostearate), polysorbate 61 (polyoxyethylene (4) sorbitan monostearate), polysorbate 65 (polyoxyethylene (20) sorbitan tristearate), polysorbate 80 (polyoxyethylene (20) sorbitan monooleate), polysorbate 81 (polyoxyethylene (5) sorbitan monooleate), polysorbate 85 (polyoxyethylene (20) sorbitan trioleate), polysorbate 120 (polyoxyethylene (20) sorbitan monoisostearate)), sorbitan fatty acid esters (such as Span®, for example sorbitan monolaurate (Span® 20), sorbitan monopalmitate (Span® 40), sorbitan monostearate (Span® 60) sorbitan tristearate (Span® 65) sorbitan monooleate (Span® 80), sorbitan sesquioleate (Span® 83), sorbitan trioleate (Span® 85), polyoxyethylene glycerol fatty acid esters (for example polyoxyethylene glycerol monostearate, polyoxyethylene glycerol ricinoleate, polyoxyethylene glycerol triricinoleate), polyoxyethylene fatty alcohol ethers (for example polyoxyethylene lauryl ether, polyoxyethylene cetyl-stearyl ether), polyoxypropylene-polyoxyethylene block copolymers (for example poloxamer), alkyl sulfates (for example sodium lauryl sulfate, sodium cetyl-stearyl sulfate), alkali soaps (for example sodium palmitate, sodium stearate) and sucrose fatty acid esters. According to one embodiment of the invention, the solubilizer is selected from the group consisting of ethanol, polysorbate 20, polyoxyethylene (8) stearate and polysorbate 80. According to one embodiment of the invention, the solubilizer is polysorbate 80.

The present invention also provides stable pharmaceutical formulations according to the invention for nasal or pharyngeal administration, wherein the optional at least one solubilizer is selected from the group consisting of ethanol, polysorbate 20, polyoxyethylene (8) stearate and polysorbate 80.

If a surfactant is present as solubilizer in the formulations according to the invention, the concentration of this surfactant is at least its critical micelle concentration (CMC) and at most the maximum approved amount for nasal or pharyngeal administration. The CMC of polysorbate 80 is 0.001% w/v and the maximum pharmaceutically approved concentration is 10% w/v. When using polysorbate 80 as solubilizer, polysorbate 80 is present in the formulations according to the invention at a concentration of 0.001-10% w/v, or 0.1-10% w/v, or 1-10% w/v or 5-10% w/v. Alternatively, polysorbate 80 may also be present in the formulations according to the invention at concentrations up to 15% w/v or up to 20% w/v.

In the context of the present invention, antioxidants are, for example, citric acid, butylhydroxyanisole, butylhydroxytoluene, EDTA, purging with nitrogen, tocopherol, ascorbic acid, glutathione, cysteine, sulfites (for example sodium sulfite, sodium hydrogensulfite), disulfites (for example sodium pyrosulfite), ascorbic acid esters or gallates. According to one embodiment of the invention, the antioxidant is selected from the group consisting of citric acid, butylhydroxyanisole, butylhydroxytoluene, EDTA and purging with nitrogen. According to one embodiment of the invention, the antioxidant is butylhydroxyanisole.

The present invention also provides stable pharmaceutical formulations according to the invention for nasal or pharyngeal administration, wherein the optional at least one antioxidant is selected from the group consisting of citric acid, butylhydroxyanisole, butylhydroxytoluene, EDTA and purging with nitrogen.

One embodiment of the present invention relates to stable pharmaceutical formulations for nasal or pharyngeal administration comprising:

a therapeutically effective amount of at least one inhibitor of the TASK-1 and/or TASK-3 channel, or a hydrate, solvate, polymorph or metabolite thereof or a pharmaceutically acceptable salt thereof in >2.5% to 100% w/v polyethylene glycol and an antioxidant and optionally at least one further auxiliary, wherein the formulation has a pH of 4 to 8.

In the context of the present invention, preservatives are, for example, phenolic substances such as phenol or cresol, alcohols such as ethanol, chlorobutanol, phenylethanol, or propylene glycol, invert soaps such as benzalkonium chloride or benzethonium chloride, benzoic acid and salts thereof, sorbic acid and salts thereof, dehydroacetic acid and sulfuric acid and salts thereof, sodium hydrogensulfite, parabens, including methylparaben and propylparaben or thiomersal. According to one embodiment of the invention, the preservative is selected from the group consisting of C₈-C₁₈ alkonium chloride, methylparaben, propylparaben, sorbic acid, chlorobutanol and benzalkonium chloride. According to one embodiment of the invention, the preservative is benzalkonium chloride.

The present invention also provides stable pharmaceutical formulations according to the invention for nasal or pharyngeal administration, wherein the optional at least one preservative is selected from the group consisting of C₈-C₁₈ alkonium chloride, methylparaben, propylparaben, sorbic acid, chlorobutanol and benzalkonium chloride.

In the context of the present invention, substances for adjusting tonicity are, for example, salts (e.g. of plasma cations with physiologically tolerable counterions), sugars (e.g. glucose, sucrose), sugar alcohols (e.g. mannitol, sorbitol), glycols (e.g. propylene glycols) and other non-ionic polyol materials.

In the context of the present invention, thickeners are, for example, natural rubbers, alginic acid, pectins, starch and starch derivatives, gelatins, poloxamers (block copolymers of ethylene oxide and propylene oxide) cellulose derivatives, acrylic acid polymers or vinyl polymers.

According to one embodiment of the present invention, the formulations according to the invention comprise at least one pH regulator as auxiliary. According to one embodiment of the present invention, the formulations according to the invention comprise at least one antioxidant as auxiliary. According to one embodiment of the present invention, the formulations according to the invention comprise at least one solubilizer as auxiliary. According to one embodiment of the present invention, the formulations according to the invention comprise at least one pH regulator and at least one solubilizer as auxiliaries. According to one embodiment of the present invention, the formulations according to the invention comprise at least one antioxidant and at least one solubilizer as auxiliaries. According to one embodiment of the present invention, the formulations according to the invention comprise at least one pH regulator, at least one solubilizer and at least one antioxidant as auxiliaries. According to one embodiment of the present invention, the formulations according to the invention comprise at least one pH regulator, at least one solubilizer, at least one antioxidant and at least one preservative as auxiliaries.

The present invention also provides stable pharmaceutical formulations according to the invention for nasal or pharyngeal administration, wherein the formulation comprises 5 to 100% w/v polyethylene glycol 400, 0 to 10% wv of a solubilizer, 0 to 95% w/v of a pH regulator and optionally at least one further auxiliary.

For the purpose of the present invention, polyethylene glycol is defined as a polyethylene glycol of the general formula (II)

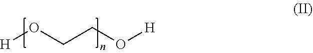

having a molar mass of 44 g/mol per repeat unit and additionally by weight of the incorporated water. The empirical formula of polyethylene glycol is C₂ₙH₄ₙ₊₂Oₙ₊₁.

In the context of the present invention, use may be made of polyethylene glycols having a mean molar mass of 200 to 3350 Da. According to a further embodiment, the present invention uses polyethylene glycols having a molecular weight of from 200 to 600 or 300 to 400 or 400. These are approved for nasal administration.

One embodiment of the present invention is a stable pharmaceutical formulation according to the invention for nasal or pharyngeal administration, wherein the formulation comprises >2.5% w/v to 100% w/v polyethylene glycol having a molecular weight of 200 to 600 and optionally comprises at least one pH regulator and optionally at least one solubilizer and optionally at least one further auxiliary.

S PEG 400 has an aver age molar mass of 400 glmol, PEG 300 has an average molar mass of 300 glmol.

In the context of the present invention, the dynamic viscosity (at 20° C.) of the formulations according to the invention is between 0.5 and 1480 mPa*s, preferably between 1.0 and 140 mPa*s. Formulations according to the invention for nasal administration by means of nasal spray preferably have a dynamic viscosity (at 20° C.) between 1.0 and 140 mPa*s. Formulations according to the invention for nasal administration by means of nasal drops preferably have a dynamic viscosity (at 20° C.) between 1.0 and 1480 mPa*s.

One embodiment of the present invention is a stable pharmaceutical formulation according to the invention for nasal or pharyngeal administration, wherein the formulation has a viscosity at 20° C. of 0.5-200 mPa*s, preferably 1-20 mPa*s.

One formulation according to the invention comprising 20% w/v PEG 400 and 10% w/v polysorbate 80 in phosphate buffer has a dynamic viscosity of ca. 6 mPa*s.

In the context of the present invention, the preferred droplet size (stated as median volume diameter) in an atomized formulation is between 5 and 300 μm, preferably between 30 and 100 μm. This is independent of whether the administration is nasal or pharyngeal.

One embodiment of the present invention is a stable pharmaceutical formulation according to the invention for nasal or pharyngeal administration, wherein the formulation is administered as a nasal spray and has a droplet size as median volume diameter of 5-300 μm, preferably 30-100 μm.

According to one embodiment of the present invention, the formulations according to the invention comprise >2.5% w/v to 100% w/v or 3% w/v to 100% w/v or 3% w/v to 90% w/v or 3% w/v to 80% w/v or 3% w/v to 70% w/v or 3% w/v to 60% w/v or 3% w/v to 50% w/v or 3% w/v to 40% w/v or 3% w/v to 30% w/v or 3% w/v to 20% w/v or 3% w/v to 10% w/v or 3% w/v to 5% w/v or 5% w/v to 100% w/v or 5% w/v to 90% w/v or 5% w/v to 80% w/v or 5% w/v to 70% w/v or 5% w/v to 60% w/v or 5% w/v to 50% w/v or 5% w/v to 40% w/v or 5% w/v to 50% w/v or 5% w/v to 20% w/v or 5% w/v to 10% w/v or 5% w/v PEG200 or PEG300 or PEG400 or PEG500 or PEG600.

According to one embodiment of the present invention, the formulations according to the invention comprise >2.5% w/v to 100% w/v or 3% w/v to 100% w/v or 3% w/v to 90% w/v or 3% w/v to 80% w/v or 3% w/v to 70% w/v or 3% w/v to 60% w/v or 3% w/v to 50% w/v or 3% w/v to 40% w/v or 3% w/v to 30% w/v or 3% w/v to 20% w/v or 3% w/v to 10% w/v or 3% w/v to 5% w/v or 5% w/v to 100% w/v or 5% w/v to 90% w/v or 5% w/v to 80% w/v or 5% w/v to 70% w/v or 5% w/v to 60% w/v or 5% w/v to 50% w/v or 5% w/v to 40% w/v or 5% w/v to 50% w/v or 5% w/v to 20% w/v or 5% w/v to 10% w/v or 5% w/v PEG400.

According to one embodiment of the present invention, the formulations according to the invention comprise the inhibitor of the TASK-1 and/or TASK-3 channel in >2.5 to 100% w/v PEG400. According to one embodiment of the present invention, the formulations according to the invention comprise the inhibitor of the TASK-1 and/or TASK-3 channel in 5 to 100% w/v PEG400. According to one embodiment of the present invention, the formulations according to the invention comprise the inhibitor of the TASK-1 and/or TASK-3 channel in 10 to 100% w/v PEG400. According to one embodiment of the present invention, the formulations according to the invention comprise the inhibitor of the TASK-1 and/or TASK-3 channel in 20 to 100% w/v PEG400. According to one embodiment of the present invention, the formulations according to the invention comprise the inhibitor of the TASK-1 and/or TASK-3 channel in 5 to 20% w/v PEG400. According to one embodiment of the present invention, the formulations according to the invention comprise the inhibitor of the TASK-1 and/or TASK-3 channel in 20% w/v PEG400.

In the context of the present invention, an active ingredient is defined as an inhibitor of the TASK-1 and/or TASK-3 channel, or a hydrate, solvate, polymorph, or metabolite thereof or a pharmaceutically acceptable salt thereof.

Stable pharmaceutical formulations according to the invention are, for example, those formulations in which the at least one inhibitor of the TASK-1 and/or TASK-3 channel is selected from the compounds described in PCT/EP2016/079973.

Stable pharmaceutical formulations according to the invention are, for example, those formulations in which the at least one inhibitor of the TASK-1 and/or TASK-3 channel is selected from compounds of the general formula (I),

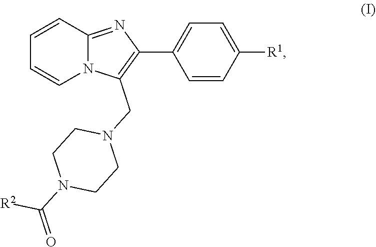

in which

R¹ represents halogen, cyano, (C₁-C₄)-alkyl, cyclopropyl or cyclobutyl

and

R² represents (C₄-C₆)-cycloalkyl in which a ring CH₂ group may be replaced by —O—

or

represents a phenyl group of the formula (a) or a pyridyl group of the formula (b)

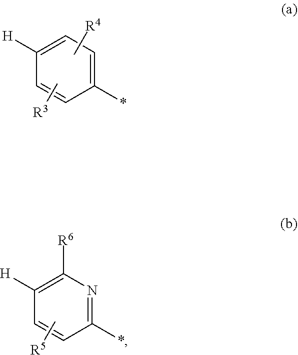

in which * marks the bond to the adjacent carbonyl group and

R³ represents fluorine, chlorine, bromine, cyano, (C₁-C₃)-alkyl or (C₁-C₃)-alkoxy,

where (C₁-C₃)-alkyl and (C₁-C₃)-alkoxy may be up to trisubstituted by fluorine,

R⁴ represents hydrogen, fluorine, chlorine, bromine or methyl,

R⁵ represents hydrogen, fluorine, chlorine, bromine or methyl,

and

R⁶ is hydrogen, (C₁-C₃)-alkoxy, cyclobutyloxy, oxetan-3-yloxy, tetrahydrofuran-3-yloxy or tetrahydro-2H-pyran-4-yloxy,

where (C₁-C₃)-alkoxy may be up to trisubstituted by fluorine,

and the salts, solvates and solvates of the salts thereof.

Stable pharmaceutical formulations according to the invention are, for example, those formulations in which the at least one inhibitor of the TASK-1 and/or TASK-3 channel is selected from compounds of the formula (I) given above, in which

R¹ represents fluorine, chlorine, bromine, methyl, isopropyl, tert-butyl or cyclopropyl and

R² represents cyclobutyl, cyclopentyl or cyclohexyl

or

represents a phenyl group of the formula (a) or a pyridyl group of the formula (b)

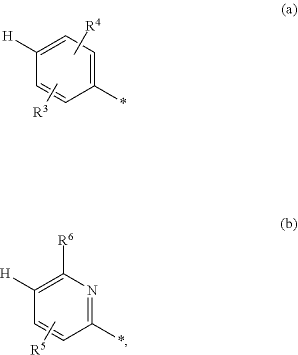

in which * marks the bond to the adjacent carbonyl group and

R³ represents fluorine, chlorine, cyano, (C₁-C₃)-alkyl, (C₁-C₃)-alkoxy or trifluoromethoxy,
R⁴ represents hydrogen, fluorine or chlorine,
R⁵ represents hydrogen, fluorine, chlorine, bromine or methyl

and

R⁶ represents hydrogen or (C₁-C₃)-alkoxy which may be up to trisubstituted by fluorine,

and the salts, solvates and solvates of the salts thereof.

Stable pharmaceutical formulations according to the invention are, for example, those formulations in which the at least one inhibitor of the TASK-1 and/or TASK-3 channel is selected from compounds of the formula (I), in which

R¹ represents chlorine or bromine,

and the salts, solvates and solvates of the salts thereof.

Stable pharmaceutical formulations according to the invention are, for example, those formulations in which the at least one inhibitor of the TASK-1 and/or TASK-3 channel is selected from compounds of the formula (I), in which

R¹ represents methyl, isopropyl, tert-butyl or cyclopropyl,

and the salts, solvates and solvates of the salts thereof.

Stable pharmaceutical formulations according to the invention are also those formulations in which the at least one inhibitor of the TASK-1 and/or TASK-3 channel is selected from compounds of the formula (I), in which

R² represents cyclobutyl, cyclopentyl or cyclohexyl,

and the salts, solvates and solvates of the salts thereof.

Stable pharmaceutical formulations according to the invention are also those formulations in which the at least one inhibitor of the TASK-1 and/or TASK-3 channel is selected from compounds of the formula (I), in which

R² represents a phenyl group of the formula (a)

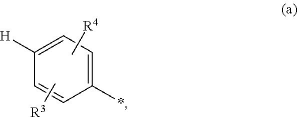

in which * marks the bond to the adjacent carbonyl group,

R³ represents fluorine, chlorine, cyano, (C₁-C₃)-alkyl or (C₁-C₃)-alkoxy

and

R⁴ represents hydrogen, fluorine or chlorine,

and the salts, solvates and solvates of the salts thereof.

Stable pharmaceutical formulations according to the invention are also those formulations in which the at least one inhibitor of the TASK-1 and/or TASK-3 channel is selected from compounds of the formula (I), in which

R² represents a pyridyl group of the formula (b)

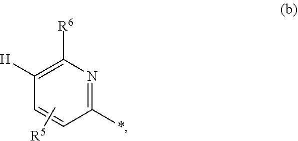

in which * marks the bond to the adjacent carbonyl group,

R⁵ represents hydrogen, chlorine or bromine

and

R⁶ represents (C₁-C₃)-alkoxy which may be up to trisubstituted by fluorine,

and the salts, solvates and solvates of the salts thereof.

Stable pharmaceutical formulations according to the invention are also those formulations in which the at least one inhibitor of the TASK-1 and/or TASK-3 channel is selected from compounds of the formula (I), in which

R¹ represents chlorine, bromine, isopropyl or cyclopropyl

and

R² represents cyclobutyl, cyclopentyl or cyclohexyl

or

represents a phenyl group of the formula (a) or a pyridyl group of the formula (b)

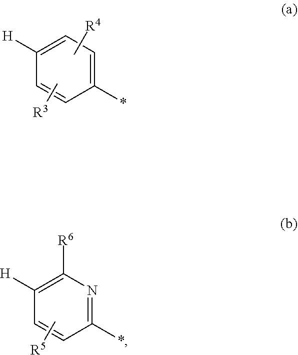

in which * marks the bond to the adjacent carbonyl group and

R³ represents fluorine, chlorine, cyano, methyl, isopropyl, methoxy or ethoxy,

R⁴ represents hydrogen, fluorine or chlorine,

R⁵ represents hydrogen, chlorine or bromine

and

R⁶ represents methoxy, difluoromethoxy, trifluoromethoxy or isopropoxy,

and the salts, solvates and solvates of the salts thereof

The individual radical definitions specified in the respective combinations or preferred combinations of radicals are, independently of the respective combinations of the radicals specified, also replaced as desired by radical definitions of other combinations.

Very particular preference is given to combinations of two or more of the abovementioned preferred ranges.

Stable pharmaceutical formulations according to the invention are also those formulations in which the at least one inhibitor of the TASK-1 and/or TASK-3 channel is selected from compounds of Table 1. The synthesis of these compounds is described in PCT/EP2016/079973.

TABLE 1
Compounds of PCT/EP2016/079973

| Example | Name |
|---|---|
| 1 | (4-{[2-(4-bromophenyl)imidazo[1,2-a]pyridin-3-yl]methyl}piperazin-1- |
| | yl)(cyclopentyl)methanone |
| 2 | (4-{[2-(4-chlorophenyl)imidazo[1,2-a]pyridin-3-yl]methyl}piperazin-1- |
| | yl)(cyclopentyl)methanone |
| 3 | (4-{[2-(4-chlorophenyl)imidazo[1,2-a]pyridin-3-yl]methyl}piperazin-1- |
| | yl)(6-methoxypyridin-2-yl)methanone |
| 4 | (4-{[2-(4-bromophenyl)imidazo[1,2-a]pyridin-3-yl]methyl}piperazin-1- |
| | yl)(2-fluorophenyl)methanone |
| 5 | (4-{[2-(4-bromophenyl)imidazo[1,2-a]pyridin-3-yl]methyl}piperazin-1- |
| | yl)(3-methoxyphenyl)methanone |
| 6 | (4-{[2-(4-bromophenyl)imidazo[1,2-a]pyridin-3-yl]methyl}piperazin-1- |
| | yl)(2-chloro-5-fluorophenyl)methanone |
| 7 | (4-{[2-(4-chlorophenyl)imidazo[1,2-a]pyridin-3-yl]methyl}piperazin-1- |
| | yl)(2-fluorophenyl)methanone |
| 8 | (4-{[2-(4-fluorophenyl)imidazo[1,2-a]pyridin-3-yl]methyl}piperazin-1- |
| | yl)(cyclohexyl)methanone |
| 9 | (4-{[2-(4-bromophenyl)imidazo[1,2-a]pyridin-3-yl]methyl}piperazin-1- |
| | yl)(cyclohexyl)methanone |
| 10 | (4-{[2-(4-bromophenyl)imidazo[1,2-a]pyridin-3-yl]methyl}piperazin-1- |
| | yl)(tetrahydrofuran-3-yl)methanone |
| 11 | (4-{[2-(4-bromophenyl)imidazo[1,2-a]pyridin-3-yl]methyl}piperazin-1- |
| | yl)(cyclobutyl)methanone |
| 12 | (4-{[2-(4-bromophenyl)imidazo[1,2-a]pyridin-3-yl]methyl}piperazin-1- |
| | yl)(2-methoxyphenyl)methanone |
| 13 | (4-{[2-(4-bromophenyl)imidazo[1,2-a]pyridin-3-yl]methyl}piperazin-1- |
| | yl)(5-fluoro-2-methoxyphenyl)methanone |
| 14 | (4-{[2-(4-bromophenyl)imidazo[1,2-a]pyridin-3-yl]methyl}piperazin-1- |
| | yl)(2-methylphenyl)methanone |
| 15 | (4-{[2-(4-bromophenyl)imidazo[1,2-a]pyridin-3-yl]methyl}piperazin-1- |
| | yl)(5-fluoro-2-methylphenyl)methanone |
| 16 | (2-chloro-5-fluorophenyl)(4-{[2-(4-chlorophenyl)imidazo[1,2-a]pyridin-3- |
| | yl]methyl}piperazin-1-yl)methanone |
| 17 | (4-{[2-(4-chlorophenyl)imidazo[1,2-a]pyridin-3-yl]methyl}piperazin-1- |
| | yl)(cyclohexyl)methanone |
| 18 | ((4-{[2-(4-chlorophenyl)imidazo[1,2-a]pyridin-3-yl]methyl}piperazin-1- |
| | yl)(cyclobutyl)methanone |
| 19 | (4-{[2-(4-chlorophenyl)imidazo[1,2-a]pyridin-3-yl]methyl}piperazin-1- |
| | yl)(3-methoxyphenyl)methanone |
| 20 | (4-{[2-(4-chlorophenyl)imidazo[1,2-a]pyridin-3-yl]methyl}piperazin-1- |
| | yl)(2-methoxyphenyl)methanone |
| 21 | (4-{[2-(4-chlorophenyl)imidazo[1,2-a]pyridin-3-yl]methyl}piperazin-1- |
| | yl)(5-fluoro-2-methoxyphenyl)methanone |
| 22 | (4-{[2-(4-chlorophenyl)imidazo[1,2-a]pyridin-3-yl]methyl}piperazin-1- |
| | yl)(2-methylphenyl)methanone |
| 23 | (4-{[2-(4-chlorophenyl)imidazo[1,2-a]pyridin-3-yl]methyl}piperazin-1- |
| | yl)(5-fluoro-2-methylphenyl)methanone |
| 24 | (4-{[2-(4-chlorophenyl)imidazo[1,2-a]pyridin-3-yl]methyl}piperazin-1- |
| | yl)[3-(trifluoromethoxy)phenyl]methanone |
| 25 | (4-{[2-(4-chlorophenyl)imidazo[1,2-a]pyridin-3-yl]methyl}piperazin-1- |
| | yl)[3-(trifluoromethyl)phenyl]methanone |
| 26 | ((4-{[2-(4-chlorophenyl)imidazo[1,2-a]pyridin-3-yl]methyl}piperazin-1- |
| | yl)(pyridin-2-yl)methanone |
| 27 | (4-{[2-(4-chlorophenyl)imidazo[1,2-a]pyridin-3-yl]methyl}piperazin-1- |
| | yl)(2-fluoro-5-methoxyphenyl)methanone |
| 28 | (4-{[2-(4-chlorophenyl)imidazo[1,2-a]pyridin-3-yl]methyl}piperazin-1- |
| | yl)(2-ethoxyphenyl)methanone |
| 29 | (2-chloro-5-methoxyphenyl)(4-{[2-(4-chlorophenyl)imidazo[1,2-a]pyridin- |
| | 3-yl]methyl}piperazin-1-yl)methanone |
| 30 | (4-{[2-(4-chlorophenyl)imidazo[1,2-a]pyridin-3-yl]methyl}piperazin-1- |
| | yl)(tetrahydro-2H-pyran-2-yl)methanone |
| 31 | (4-{[2-(4-chlorophenyl)imidazo[1,2-a]pyridin-3-yl]methyl}piperazin-1- |
| | yl)(3-isopropoxyphenyl)methanone |
| 32 | 2-[(4-{[2-(4-chlorophenyl)imidazo[1,2-a]pyridin-3-yl]methyl}piperazin-1- |
| | yl)carbonyl]benzonitrile |
| 33 | (4-{[2-(4-chlorophenyl)imidazo[1,2-a]pyridin-3-yl]methyl}piperazin-1- |
| | yl)(3-isopropylphenyl)methanone |
| 34 | (4-{[2-(4-chlorophenyl)imidazo[1,2-a]pyridin-3-yl]methyl}piperazin-1- |
| | yl)(2-isopropylphenyl)methanone |
| 35 | (4-{[2-(4-chlorophenyl)imidazo[1,2-a]pyridin-3-yl]methyl}piperazin-1- |
| | yl)(tetrahydrofuran-2-yl)methanone |
| 36 | (3-chlorophenyl)(4-{[2-(4-chlorophenyl)imidazo[1,2-a]pyridin-3- |
| | yl]methyl}piperazin-1-yl)methanone |
| 37 | (2-chlorophenyl)(4-{[2-(4-chlorophenyl)imidazo[1,2-a]pyridin-3- |
| | yl]methyl}piperazin-1-yl)methanone |
| 38 | (4-{[2-(4-chlorophenyl)imidazo[1,2-a]pyridin-3-yl]methyl}piperazin-1- |
| | yl)[6-(2,2,2-trifluoroethoxy)pyridin-2-yl]methanone |
| 39 | (4-{[2-(4-chlorophenyl)imidazo[1,2-a]pyridin-3-yl]methyl}piperazin-1- |
| | yl)(6-isopropoxypyridin-2-yl)methanone |
| 40 | (4-{[2-(4-chlorophenyl)imidazo[1,2-a]pyridin-3-yl]methyl}piperazin-1- |
| | yl)(6-methoxy-4-methylpyridin-2-yl)methanone |
| 41 | (4-{[2-(4-chlorophenyl)imidazo[1,2-a]pyridin-3-yl]methyl}piperazin-1- |
| | yl)[6-(cyclobutyloxy)pyridin-2-yl]methanone |
| 42 | (3-bromo-6-methoxypyridin-2-yl)(4-{[2-(4-chlorophenyl)imidazo[1,2- |
| | a]pyridin-3-yl]methyl}piperazin-1-yl)methanone |
| 43 | (3-chloro-6-methoxypyridin-2-yl)(4-{[2-(4-chlorophenyl)imidazo[1,2- |
| | a]pyridin-3-yl]methyl}piperazin-1-yl)methanone |
| 44 | (4-{[2-(4-chlorophenyl)imidazo[1,2-a]pyridin-3-yl]methyl}piperazin-1- |
| | yl)[6-(difluoromethoxy)pyridin-2-yl]methanone |
| 45 | (4-{[2-(4-chlorophenyl)imidazo[1,2-a]pyridin-3-yl]methyl}piperazin-1- |
| | yl)(6-ethoxypyridin-2-yl)methanone |
| 46 | (4-{[2-(4-chlorophenyl)imidazo[1,2-a]pyridin-3-yl]methyl}piperazin-1- |
| | yl)[6-(tetrahydro-2H-pyran-4-yloxy)pyridin-2-yl]methanone |
| 47 | (4-{[2-(4-bromophenyl)imidazo[1,2-a]pyridin-3-yl]methyl}piperazin-1- |
| | yl)(6-methoxypyridin-2-yl)methanone |
| 48 | (4-{[2-(4-fluorophenyl)imidazo[1,2-a]pyridin-3-yl]methyl}piperazin-1- |
| | yl)(cyclopentyl)methanone |
| 49 | (4-{[2-(4-fluorophenyl)imidazo[1,2-a]pyridin-3-yl]methyl}piperazin-1- |
| | yl)(cyclobutyl)methanone |
| 50 | (5-fluoro-2-methoxyphenyl)(4-{[2-(4-fluorophenyl)imidazo[1,2-a]pyridin-3- |
| | yl]methyl}piperazin-1-yl)methanone |
| 51 | (2-chloro-5-fluorophenyl)(4-{[2-(4-fluorophenyl)imidazo[1,2-a]pyridin-3- |
| | yl]methyl}piperazin-1-yl)methanone |
| 52 | (4-{[2-(4-fluorophenyl)imidazo[1,2-a]pyridin-3-yl]methyl}piperazin-1- |
| | yl)(2-methoxyphenyl)methanone |
| 53 | (2-fluorophenyl)(4-{[2-(4-isopropylphenyl)imidazo[1,2-a]pyridin-3- |
| | yl]methyl}piperazin-1-yl)methanone |
| 54 | cyclopentyl(4-{[2-(4-isopropylphenyl)imidazo[1,2-a]pyridin-3- |
| | yl]methyl}piperazin-1-yl)methanone |
| 55 | (4-{[2-(4-isopropylphenyl)imidazo[1,2-a]pyridin-3-yl]methyl}piperazin-1- |
| | yl)(6-methoxypyridin-2-yl)methanone |
| 56 | cyclopentyl(4-{[2-(4-methylphenyl)imidazo[1,2-a]pyridin-3- |
| | yl]methyl}piperazin-1-yl)methanone |
| 57 | cyclohexyl(4-{[2-(4-methylphenyl)imidazo[1,2-a]pyridin-3- |
| | yl]methyl}piperazin-1-yl)methanone |
| 58 | (2-methoxyphenyl)(4-{[2-(4-methylphenyl)imidazo[1,2-a]pyridin-3- |
| | yl]methyl}piperazin-1-yl)methanone |
| 59 | (6-methoxypyridin-2-yl)(4-{[2-(4-methylphenyl)imidazo[1,2-a]pyridin-3- |
| | yl]methyl}piperazin-1-yl)methanone |
| 60 | (4-(3-{[4-(2-fluorobenzoyl)piperazin-1-yl]methyl}imidazo[1,2-a]pyridin-2- |
| | yl)benzonitrile |
| 61 | 4-[3-({4-[(6-methoxypyridin-2-yl)carbonyl]piperazin-1- |
| | yl}methyl)imidazo[1,2-a]pyridin-2-yl]benzonitrile |
| 62 | 4-(3-{[4-(cyclopentylcarbonyl)piperazin-1-yl]methyl}imidazo[1,2-a]pyridin- |
| | 2-yl)benzonitrile |
| 63 | 4-(3-{[4-(cyclohexylcarbonyl)piperazin-1-yl]methyl}imidazo[1,2-a]pyridin- |
| | 2-yl)benzonitrile |
| 64 | (4-{[2-(4-tert-butylphenyl)imidazo[1,2-a]pyridin-3-yl]methyl}piperazin-1- |
| | yl)(6-methoxypyridin-2-yl)methanone |
| 65 | (4-{[2-(4-tert-butylphenyl)imidazo[1,2-a]pyridin-3-yl]methyl}piperazin-1- |
| | yl)(2-fluorophenyl)methanone |
| 66 | (4-{[2-(4-tert-butylphenyl)imidazo[1,2-a]pyridin-3-yl]methyl}piperazin-1- |
| | yl)(cyclopentyl)methanone |
| 67 | (4-{[2-(4-chlorophenyl)imidazo[1,2-a]pyridin-3-yl]methyl}piperazin-1- |
| | yl)[6-(trifluoromethoxy)pyridin-2-yl]methanone |
| 68 | (4-{[2-(4-chlorophenyl)imidazo[1,2-a]pyridin-3-yl]methyl}piperazin-1- |
| | yl)(3-fluoro-6-methoxypyridin-2-yl)methanone |
| 69 | (4-{[2-(4-cyclopropylphenyl)imidazo[1,2-a]pyridin-3-yl]methyl}piperazin- |
| | 1-yl)(2-fluorophenyl)methanone |
| 70 | 4-(3-{[4-(2-fluoro-5-methoxybenzoyl)piperazin-1-yl]methyl}imidazo[1,2- |
| | a]pyridin-2-yl)benzonitrile |
| 71 | 4-[3-({4-[(6-methoxy-3-methylpyridin-2-yl)carbonyl]piperazin-1- |
| | yl}methyl)imidazo[1,2-a]pyridin-2-yl]benzonitrile |
| 72 | (4-{[2-(4-chlorophenyl)imidazo[1,2-a]pyridin-3-yl]methyl}piperazin-1- |
| | yl)(6-methoxy-3-methylpyridin-2-yl)methanone |
| 73 | (4-{[2-(4-tert-butylphenyl)imidazo[1,2-a]pyridin-3-yl]methyl}piperazin-1- |
| | yl)(6-methoxy-3-methylpyridin-2-yl)methanone |
| 74 | (4-{[2-(4-bromophenyl)imidazo[1,2-a]pyridin-3-yl]methyl}piperazin-1- |
| | yl)(6-methoxy-3-methylpyridin-2-yl)methanone |

Stable pharmaceutical formulations according to the invention are also those formulations in which the at least one inhibitor of the TASK-1 and/or TASK-3 channel is selected from the group consisting of

| Example | Name |
|---|---|
| 1 | (4-{[2-(4-Bromophenyl)imidazo[1,2-a]pyridin-3- |
| | yl]methyl}piperazin-1-yl)(cyclopentyl)methanone |
| 2 | (4-{[2-(4-Chlorophenyl)imidazo[1,2-a]pyridin-3- |
| | yl]methyl}piperazin-1-yl)(cyclopentyl)methanone |
| 3 | (4-{[2-(4-Chlorophenyl)imidazo[1,2-a]pyridin-3- |
| | yl]methyl}piperazin-1-yl)(6-methoxypyridin-2-yl)methanone |
| 4 | (4-{[2-(4-Bromophenyl)imidazo[1,2-a]pyridin-3- |
| | yl]methyl}piperazin-1-yl)(2-fluorophenyl)methanone |

and the salts, solvates and solvates of the salts thereof.

Stable pharmaceutical formulations according to the invention are also those formulations in which the at least one inhibitor of the TASK-1 and/or TASK-3 channel is (4-{[2-(4-chlorophenyl)imidazo[1,2-a]pyridin-3-yl]methyl}piperazin-1-yl)(6-methoxypyridin-2-yl)methanone.

A further embodiment of the present invention are the stable pharmaceutical formulations according to the invention for nasal or pharyngeal administration for the treatment and/or prevention of diseases.

A further embodiment of the present invention are the stable pharmaceutical formulations according to the invention for nasal or pharyngeal administration for use in a method for the treatment and/or prevention of respiratory disorders, sleep-related respiratory disorders, obstructive sleep apnoeas, central sleep apnoeas, snoring, cardiac arrhythmias, arrhythmias, neurodegenerative disorders, neuroinflammatory disorders and neuroimmunological disorders.

A further embodiment of the present invention are the stable pharmaceutical formulations according to the invention for nasal or pharyngeal administration for use in a method for the treatment and/or prevention of respiratory disorders, sleep-related respiratory disorders, obstructive sleep apnoeas, central sleep apnoeas, snoring, cardiac arrhythmias, arrhythmias, neurodegenerative disorders, neuroinflammatory disorders and neuroimmunological disorders, wherein the nasal or pharyngeal administration is aided by nasal sprays, nasal drops, nasal solutions, powder inhalers, nebulizers, metered dose aerosols or semisolid gels.

A further embodiment of the present invention are the stable pharmaceutical formulations according to the invention for nasal or pharyngeal administration for use in a method for the treatment and/or prevention of respiratory disorders, sleep-related respiratory disorders, obstructive sleep apnoeas, central sleep apnoeas, snoring, cardiac arrhythmias, arrhythmias, neurodegenerative disorders, neuroinflammatory disorders and neuroimmunological disorders, wherein the duration of action is at least 3 hours.

A further embodiment of the present invention are the stable pharmaceutical formulations according to the invention for nasal or pharyngeal administration for use in a method for the treatment and/or prevention of respiratory disorders, sleep-related respiratory disorders, obstructive sleep apnoeas, central sleep apnoeas, snoring, cardiac arrhythmias, arrhythmias, neurodegenerative disorders, neuroinflammatory disorders and neuroimmunological disorders, wherein the duration of action is at least 4 hours.

A further embodiment of the present invention are the stable pharmaceutical formulations according to the invention for nasal or pharyngeal administration for use in a method for the treatment and/or prevention of respiratory disorders, sleep-related respiratory disorders, obstructive sleep apnoeas, central sleep apnoeas, snoring, cardiac arrhythmias, arrhythmias, neurodegenerative disorders, neuroinflammatory disorders and neuroimmunological disorders, wherein the duration of action is at least 5 hours.

A further embodiment of the present invention are the stable pharmaceutical formulations according to the invention for nasal or pharyngeal administration for use in a method for the treatment and/or prevention of obstructive sleep apnoeas or snoring, comprising:

a therapeutically effective amount of the inhibitor of the TASK-1 and/or TASK-3 channel 4-{[2-(4-chlorophenyl)imidazo[1,2-a] pyridin-3-yl]methyl}piperazin-1-yl)(6-methoxy-pyridin-2-yl)methanone or a hydrate, solvate, polymorph or metabolite thereof or a pharmaceutically acceptable salt thereof in 20% to 100% w/v PEG400 and 0 to 10% w/v polysorbate 80 and 0 to 80% w/v of a phosphate buffer having a pH of 7, and optionally at least one further auxiliary, wherein the duration of action of the stable pharmaceutical formulation after nasal or pharyngeal administration is at least 3 hours or at least 4 hours or at least 5 hours or at least 6 hours or at least 7 hours or at least 8 hours.

A further embodiment of the present invention are the stable pharmaceutical formulations according to the invention for nasal or pharyngeal administration for use in a method for the treatment and/or prevention of obstructive sleep apnoeas or snoring, comprising a therapeutically effective amount of the inhibitor of the TASK-1 and/or TASK-3 channel 4-{[2-(4-chlorophenyl)imidazo[1,2-a] pyridin-3-yl]methyl}piperazin-1-yl)(6-methoxypyridin-2-yl)methanone or a hydrate, solvate, polymorph or metabolite thereof or a pharmaceutically acceptable salt thereof in 20% to 100% w/v PEG400 and 0 to 10% w/v polysorbate 80 and 0 to 80% w/v of a phosphate buffer having a pH of 7, and optionally at least one further auxiliary, wherein the duration of action of the stable pharmaceutical formulation after nasal or pharyngeal administration is at least 3 hours.

A further embodiment of the present invention are the stable pharmaceutical formulations according to the invention for nasal or pharyngeal administration for use in a method for the treatment and/or prevention of obstructive sleep apnoeas or snoring, comprising:

a therapeutically effective amount of the inhibitor of the TASK-1 and/or TASK-3 channel 4-{[2-(4-chlorophenyl)imidazo[1,2-a] pyridin-3-yl]methyl}piperazin-1-yl)(6-methoxypyridin-2-yl)methanone or a hydrate, solvate, polymorph or metabolite thereof or a pharmaceutically acceptable salt thereof in 20% to 100% w/v PEG400 and 0 to 10% w/v polysorbate 80 and 0 to 80% w/v of a phosphate buffer having a pH of 7, and optionally at least one further auxiliary, wherein the duration of action of the stable pharmaceutical formulation after nasal or pharyngeal administration is at least 4 hours.

A further embodiment of the present invention are the stable pharmaceutical formulations according to the invention for nasal or pharyngeal administration for use in a method for the treatment and/or prevention of obstructive sleep apnoeas or snoring, comprising:

a therapeutically effective amount of the inhibitor of the TASK-1 and/or TASK-3 channel 4-{[2-(4-chlorophenyl)imidazo[1,2-a] pyridin-3-yl]methyl}piperazin-1-yl)(6-methoxy-pyridin-2-yl)methanone or a hydrate, solvate, polymorph or metabolite thereof or a pharmaceutically acceptable salt thereof in 20% to 100% w/v PEG400 and 0 to 10% w/v polysorbate 80 and 0 to 80% w/v of a phosphate buffer having a pH of 7, and optionally at least one further auxiliary, wherein the duration of action of the stable pharmaceutical formulation after nasal or pharyngeal administration is at least 5 hours.

The formulations of the invention can be used alone or, if required, in combination with one or more other pharmacologically active substances, provided that this combination does not lead to undesirable and unacceptable side effects. The present invention therefore further provides medicaments comprising at least one of the formulations of the invention and one or more further active ingredients, especially for treatment and/or prevention of the aforementioned disorders. Preferred examples of combination active ingredients suitable for this purpose include:
respiratory stimulants, by way of example and with preference theophylline, doxapram, nikethamide or caffeine;
psychostimulants, by way of example and with preference modafinil or armodafinil;
amphetamines and amphetamine derivatives, by way of example and with preference amphetamine, metamphetamine or methylphenidate;
serotonin reuptake inhibitors, by way of example and with preference fluoxetine, paroxetine, citalopram, escitalopram, sertraline, fluvoxamine or trazodone;
serotonin precursors, by way of example and with preference L-tryptophan;
selective serotonin noradrenaline reuptake inhibitors, by way of example and with preference venlafaxine or duloxetine;
noradrenergic and specifically serotonergic antidepressants, by way of example and with preference mirtazapine;
selective noradrenaline reuptake inhibitors, by way of example and with preference reboxetine;
tricyclic antidepressants, by way of example and with preference amitriptyline, protriptyline, doxepine, trimipramine, imipramine, clomipramine or desipramine;
alpha2-adrenergic agonists, by way of example and with preference clonidine;
GABA agonists, by way of example and with preference baclofen;
alpha sympathomimetics, by way of example and with preference xylometazoline, oxymetazoline, phenylephrine, naphazoline, tetryzoline or tramazoline;
glucocorticoids, by way of example and with preference fluticasone, budesonide, beclometasone, mometasone, tixocortol or triamcinolone;
cannabinoid receptor agonists;
carboanhydrase inhibitors, by way of example and with preference acetazolamide, methazolamide or diclofenamide;
opioid and benzodiazepine receptor antagonists, by way of example and with preference flumazenil, naloxone or naltrexone;
cholinesterase inhibitors, by way of example and with preference neostigmine, pyridostigmine, physostigmine, donepezil, galantamine or rivastigmine;
N-methyl-D-aspartate and glutamate antagonists, by way of example and with preference amantadine, memantine or sabeluzole;
nicotine receptor agonists;
leukotriene receptor antagonists, by way of example and with preference montelukast or tripelukast;
dopamine receptor antagonists, by way of example and with preference dromperidone, metoclopramide or benzamide, butyrophenone or phenothiazine derivatives;
appetite suppressants, by way of example and with preference sibutramine, topiramate, phentermine, lipase inhibitors or cannabinoid receptor antagonists;
proton pump inhibitors, by way of example and with preference pantoprazole, omeprazole, esomeprazole, lansoprazole or rabeprazole;
organic nitrates and NO donors, for example sodium nitroprusside, nitroglycerin, isosorbide mononitrate, isosorbide dinitrate, molsidomine or SIN-1, and inhaled NO;
compounds which inhibit the degradation of cyclic guanosine monophosphate (cGMP) and/or cyclic adenosine monophosphate (cAMP), for example inhibitors of phosphodiesterases (PDE) 1, 2, 3, 4 and/or 5, especially PDE 5 inhibitors such as sildenafil, vardenafil, tadalafil, udenafil, dasantafil, avanafil, mirodenafil or lodenafil;
NO- and haem-independent activators of soluble guanylate cyclase (sGC), such as in particular the compounds described in WO 01/19355, WO 01/19776, WO 01/19778, WO 01/19780, WO 02/070462 and WO 02/070510;
NO-independent but haem-dependent stimulators of soluble guanylate cyclase (sGC), such as in particular riociguat, vericiguat and the compounds described in WO 00/06568, WO 00/06569, WO 02/42301, WO 03/095451, WO 2011/147809, WO 2012/004258, WO 2012/028647 and WO 2012/059549;
prostacyclin analogues and IP receptor agonists, by way of example and with preference iloprost, beraprost, treprostinil, epoprostenol or selexipag;
endothelin receptor antagonists, by way of example and with preference bosentan, darusentan, ambrisentan or sitaxsentan;
compounds which inhibit human neutrophile elastase (HNE), by way of example and with preference sivelestat or DX-890 (reltran);
compounds which inhibit the degradation and alteration of the extracellular matrix, by way of example and with preference inhibitors of the matrix metalloproteases (MMPs), especially inhibitors of stromelysin, collagenases, gelatinases and aggrecanases (in this context particularly of MMP-1, MMP-3, MMP-8, MMP-9, MMP-10, MMP-11 and MMP-13) and of metalloelastase (MMP-12);
compounds which block the binding of serotonin to its receptors, by way of example and with preference antagonists of the 5-HT_{2B} receptor such as PRX-08066;
antagonists of growth factors, cytokines and chemokines, by way of example and with preference antagonists of TGF-β, CTGF, IL-1, IL-4, IL-5, IL-6, IL-8, IL-13 and integrins;
Rho kinase-inhibiting compounds, by way of example and with preference fasudil, Y-27632, SLx-2119, BF-66851, BF-66852, BF-66853, KI-23095 or BA-1049;
compounds which influence the energy metabolism of the heart, by way of example and with preference etomoxir, dichloroacetate, ranolazine or trimetazidine;
compounds which inhibit the signal transduction cascade, by way of example and with preference from the group of the kinase inhibitors, in particular from the group of the tyrosine kinase and/or serine/threonine kinase inhibitors, by way of example and with preference nintedanib, dasatinib, nilotinib, bosutinib, regorafenib, sorafenib, sunitinib, cediranib, axitinib, telatinib, imatinib, brivanib, pazopanib, vatalanib, gefitinib, erlotinib, lapatinib, canertinib, lestaurtinib, pelitinib, semaxanib or tandutinib;
anti-obstructive agents as used, for example, for treatment of chronic obstructive pulmonary disease (COPD) or bronchial asthma, by way of example and with preference from the group of the inhalatively or systemically administered agonists of the beta-adrenergic receptor (beta-mimetics) and the inhalatively administered anti-muscarinergic substances;
antiinflammatory, immunomodulating, immunosuppressive and/or cytotoxic agents, by way of example and with preference from the group of the systemically or inhalatively administered corticosteroids and also dimethyl fumarate, fingolimod, glatiramer acetate, β-interferons, natalizumab, teriflunomide, mitoxantrone, immunoglobulins, acetylcysteine, montelukast, tripelukast, azathioprine, cyclophosphamide, hydroxycarbamide, azithromycin, interferon-γ, pirfenidone or etanercept;
antifibrotic agents, by way of example and with preference lysophosphatidic acid receptor 1 (LPA-1) antagonists, CTGF inhibitors, IL-4 antagonists, IL-13 antagonists, TGF-β antagonists or pirfenidone;
antithrombotic agents, by way of example and with preference from the group of platelet aggregation inhibitors, the anticoagulants and the profibrinolytic substances;
hypotensive active ingredients, by way of example and with preference from the group of the calcium antagonists, angiotensin AII antagonists, ACE inhibitors, vasopeptidase inhibitors, endothelin antagonists, renin inhibitors, alpha receptor blockers, beta receptor blockers, mineralocorticoid receptor antagonists and also the diuretics; and/or
active ingredients that alter lipid metabolism, by way of example and with preference from the group of the thyroid receptor agonists, cholesterol synthesis inhibitors, by way of example and preferably, HMG-CoA reductase inhibitors or squalene synthesis inhibitors, the ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbers, bile acid reabsorption inhibitors and lipoprotein(a) antagonists.

In a preferred embodiment of the invention, the formulations of the invention are administered in combination with a beta-adrenergic receptor agonist, by way of example and with preference albuterol, isoproterenol, metaproterenol, terbutalin, fenoterol, formoterol, reproterol, salbutamol or salmeterol.

In a preferred embodiment of the invention, the formulations of the invention are administered in combination with an antimuscarinergic substance, by way of example and with preference ipratropium bromide, tiotropium bromide or oxitropium bromide.

In a preferred embodiment of the invention, the formulations of the invention are administered in combination with a corticosteroid, by way of example and with preference prednisone, prednisolone, methylprednisolone, triamcinolone, dexamethasone, betamethasone, beclometasone, flunisolide, budesonide or fluticasone.

Antithrombotic agents are preferably understood to mean compounds from the group of the platelet aggregation inhibitors, the anticoagulants and the profibrinolytic substances.

In a preferred embodiment of the invention, the formulations according to the invention are administered in combination with a platelet aggregation inhibitor, by way of example and with preference aspirin, clopidogrel, ticlopidine or dipyridamole.

In a preferred embodiment of the invention, the formulations of the invention are administered in combination with a thrombin inhibitor, by way of example and with preference ximelagatran, melagatran, dabigatran, bivalirudin or clexane.

In a preferred embodiment of the invention, the formulations of the invention are administered in combination with a GPIIb/IIIa antagonist, by way of example and with preference tirofiban or abciximab.

In a preferred embodiment of the invention, the formulations of the invention are administered in combination with a factor Xa inhibitor, by way of example and with preference rivaroxaban, apixaban, fidexaban, razaxaban, fondaparinux, idraparinux, DU-176b, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the formulations according to the invention are administered in combination with heparin or with a low molecular weight (LMW) heparin derivative.

In a preferred embodiment of the invention, the formulations according to the invention are administered in combination with a vitamin K antagonist, by way of example and with preference coumarin.

Hypotensive agents are preferably understood to mean compounds from the group of the calcium antagonists, angiotensin AII antagonists, ACE inhibitors, endothelin antagonists, renin inhibitors, alpha receptor blockers, beta receptor blockers, mineralocorticoid receptor antagonists, and the diuretics.

In a preferred embodiment of the invention, the formulations according to the invention are administered in combination with a calcium antagonist, by way of example and with preference nifedipine, amlodipine, verapamil or diltiazem.

In a preferred embodiment of the invention, the formulations according to the invention are administered in combination with an alpha-1 receptor blocker, by way of example and with preference prazosin.

In a preferred embodiment of the invention, the formulations according to the invention are administered in combination with a beta receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the formulations according to the invention are administered in combination with an angiotensin AII antagonist, preferred examples being losartan, candesartan, valsartan, telmisartan or embusartan.

In a preferred embodiment of the invention, the formulations according to the invention are administered in combination with an ACE inhibitor, by way of example and with preference enalapril, captopril, lisinopril, ramipril, delapril, fosinopril, quinopril, perindopril or trandopril.

In a preferred embodiment of the invention, the formulations according to the invention are administered in combination with an endothelin antagonist, by way of example and with preference bosentan, darusentan, ambrisentan or sitaxsentan.

In a preferred embodiment of the invention, the formulations according to the invention are administered in combination with a renin inhibitor, by way of example and with preference aliskiren, SPP-600 or SPP-800.

In a preferred embodiment of the invention, the formulations according to the invention are administered in combination with a mineralocorticoid receptor antagonist, by way of example and with preference spironolactone, eplerenone or finerenone.

In a preferred embodiment of the invention, the formulations according to the invention are administered in combination with a diuretic, by way of example and with preference furosemide, bumetanide, torsemide, bendroflumethiazide, chlorothiazide, hydrochlorothiazide, hydroflumethiazide, methyclothiazide, polythiazide, trichlormethiazide, chlorthalidone, indapamide, metolazone, quinethazone, acetazolamide, dichlorphenamide, methazolamide, glycerol, isosorbide, mannitol, amiloride or triamterene.

Lipid metabolism modifiers are preferably understood to mean compounds from the group of the CETP inhibitors, thyroid receptor agonists, cholesterol synthesis inhibitors such as HMG-CoA reductase inhibitors or squalene synthesis inhibitors, the ACAT inhibitors, MTP inhibitors, PPAR-alpha, PPAR-gamma and/or PPAR-delta agonists, cholesterol absorption inhibitors, polymeric bile acid adsorbers, bile acid reabsorption inhibitors, lipase inhibitors and the lipoprotein(a) antagonists.

In a preferred embodiment of the invention, the formulations according to the invention are administered in combination with a CETP inhibitor, by way of example and with preference torcetrapib (CP-529 414), HT-705 or CETP vaccine (Avant).

In a preferred embodiment of the invention, the formulations according to the invention are administered in combination with a thyroid receptor agonist, by way of example and with preference D-thyroxine, 3,5,3′-triiodothyronine (T3), CGS 23425 or axitirome (CGS 26214).

In a preferred embodiment of the invention, the formulations according to the invention are administered in combination with an HMG-CoA reductase inhibitor from the class of statins, by way of example and with preference lovastatin, simvastatin, pravastatin, fluvastatin, atorvastatin, rosuvastatin or pitavastatin.

In a preferred embodiment of the invention, the formulations according to the invention are administered in combination with a squalene synthesis inhibitor, by way of example and with preference BMS-188494 or TAK-475.

In a preferred embodiment of the invention, the formulations according to the invention are administered in combination with an ACAT inhibitor, by way of example and with preference avasimibe, melinamide, pactimibe, eflucimibe or SMP-797.

In a preferred embodiment of the invention, the formulations according to the invention are administered in combination with an MTP inhibitor, by way of example and with preference implitapide, BMS-201038, R-103757 or JTT-130.

In a preferred embodiment of the invention, the formulations according to the invention are administered in combination with a PPAR-gamma agonist, by way of example and with preference pioglitazone or rosiglitazone.

In a preferred embodiment of the invention, the formulations according to the invention are administered in combination with a PPAR-delta agonist, by way of example and with preference GW 501516 or BAY 68-5042.

In a preferred embodiment of the invention, the formulations according to the invention are administered in combination with a cholesterol absorption inhibitor, by way of example and with preference ezetimibe, tiqueside or pamaqueside.

In a preferred embodiment of the invention, the formulations according to the invention are administered in combination with a lipase inhibitor, by way of example and with preference orlistat.

In a preferred embodiment of the invention, the formulations according to the invention are administered in combination with a polymeric bile acid adsorber, by way of example and with preference cholestyramine, colestipol, colesolvam, CholestaGel or colestimide.

In a preferred embodiment of the invention, the formulations according to the invention are administered in combination with a bile acid reabsorption inhibitor, by way of example and with preference ASBT (=IBAT) inhibitors, for example AZD-7806, S-8921, AK-105, BARI-1741, SC-435 or SC-635.

In a preferred embodiment of the invention, the formulations according to the invention are administered in combination with a lipoprotein(a) antagonist, by way of example and with preference gemcabene calcium (CI-1027) or nicotinic acid.

Particular preference is given to combinations of the formulations according to the invention with one or more further active ingredients selected from the group consisting of respiratory stimulants, psychostimulants, serotonin reuptake inhibitors, noradrenergic, serotonergic and tricyclic antidepressants, sGC stimulators, mineralocorticoid receptor antagonists, antiinflammatory agents, immunomodulators, immunosuppressants and cytotoxic agents.

If required, the formulations according to the invention can also be employed in conjunction with the use of one or more medical technical devices or auxiliaries, provided that this does not lead to unwanted and unacceptable side-effects. Medical devices and auxiliaries suitable for such a combined application are, by way of example and with preference:
devices for positive airway pressure ventilation, by way of example and with preference CPAP (continuous positive airway pressure) devices, BiPAP (bilevel positive airway pressure) devices and IPPV (intermittent positive pressure ventilation) devices;
neurostimulators of the Nervus hypoglossus;
intraoral auxiliaries, by way of example and with preference protrusion braces;
nasal disposable valves;
nasal stents.

In one embodiment, the dosage in the case of intranasal administration is about 0.1 μg to 500 μg per day.

In a further embodiment, the dosage in the case of intranasal administration is about 1 μg to 250 μg per day. In a further embodiment, the dosage in the case of intranasal administration is about 1 μg to 120 μg per day. In a further embodiment, the dose of about 0.1 μg to 500 μg per day, or of about 1 μg to 250 μg per day, or of about 1 μg to 120 μg per day, is administered once daily by the intranasal route before sleeping. In one embodiment, the dose of about 0.1 μg to 500 μg per day, or of about 1 μg to 250 μg per day, or of about 1 μg to 120 μg per day, is administered once daily with half to each nostril. In one embodiment, the dose of about 0.1 μg to 500 μg per day, or of about 1 μg to 250 μg per day, or of about 1 μg to 120 μg per day, is administered once daily with half to each nostril before sleeping.

It may nevertheless be necessary in some cases to deviate from the stated amounts, and specifically as a function of body weight, route of administration, individual response to the active ingredient, nature of the preparation and time at which or interval over which administration takes place. Thus in some cases it may be sufficient to manage with less than the abovementioned minimum amount, while in other cases the upper limit mentioned must be exceeded. In the case of administration of greater amounts, it may be advisable to divide them into several individual doses over the day.

### Assessment of the Pharmacological Activity List of Abbreviations

| | |
|---|---|
| AHI | Apnoea-Hypopnoea Index |
| Na-CMC | Na carboxymethyl cellulose |
| CMC | Critical micelle concentration |
| CPAP system | Continuous positive airway pressure system |
| EDTA | Ethylenediaminetetraacetic acid |
| EMG | Electromyogram |
| mPa * s | Millipascal seconds |
| OSA | Obstructive sleep apnoea |
| PEG | Polyethylene glycol |
| TASK | TWIK-related acid-sensitive K⁺ channel |

The pharmacological activity of the inhibitors of the TASK-1 and/or TASK-3 channel present in the formulations according to the invention was demonstrated by in vitro experiments in PCT/EP2016/079973.

The pharmacological activity of the formulations according to the invention can be demonstrated by in vivo studies as known to the person skilled in the art. The application examples which follow describe the biological action of the compounds of the invention, without restricting the invention to these examples.

### Animal Model of Obstructive Sleep Apnoea in the Pig

The effects of the formulations according to the invention of the inhibitors of TASK-1 and/or TASK-3 channels on the activation threshold of the genioglossus muscle by negative pressure and the collapsibility of the upper airways were investigated in a pig model for obstructive sleep apnoea.

Using negative pressure, it is possible to induce collapse and thus obstruction of the upper airways in anaesthetized, spontaneously breathing pigs [Wirth et al., Sleep 36, 699-708 (2013)].

German Landrace pigs were used for the model. Since the nasal axis is in an almost vertical position in humans in a horizontal sleeping position, the pigs in the experiments were fixed in a sitting position (70 degrees), wherein the nose pointed upwards. After nasal administration, the formulation therefore flowed downwards over all regions of the upper airways. The pigs were anaesthetized and tracheotomized. One cannula each was inserted into the rostral and the caudal part of the trachea. Using a T connector, the rostral cannula was connected on the one hand to a device generating negative pressure and on the other hand to the caudal cannula. Using a T connector, the caudal cannula was connected to the rostral cannula and to a tube which allowed spontaneous breathing circumventing the upper airways. By appropriate closing and opening of the tubes it was thus possible for the pig to change from normal nasal breathing to breathing via the caudal cannula during the time when the upper airways were isolated and were connected to the device for generating negative pressure. The muscle activity of the Musculus genioglossus was recorded by electromyogram (EMG).

At certain points in time, the collapsibility of the upper airways was tested by having the pig breathe via the caudal cannula and applying negative pressures of −50, −100 and −150 mbar (corresponding to −50, −100 and −150 cm water column (cm H₂O)) to the upper airways. This caused the upper airways to collapse, which manifested itself in an interruption of the airflow and a pressure drop in the tube system. This test was conducted prior to administration of the test substance and at certain intervals after administration of the test substance. An appropriately effective test substance could prevent this collapse of the airways in the inspiratory phase.

After changeover from nasal breathing to breathing via the caudal cannula, it was not possible to measure any EMG activity of the Musculus genioglossus in the anaesthetized pig. As a further test, the negative pressure at which EMG activity restarted was then determined. This threshold value was, if a test substance was effective, shifted to more positive values. The test was likewise conducted prior to the administration of the test substance and at certain intervals after the administration of the test substance. The test substance was administered by the nasal route.

The results shown in the tables which follow were conducted with the compounds listed in Table 1 as Example 1, Example 3 and Example 4. Unless stated otherwise, the data were measured at a negative pressure of −100 mbar (corresponding to −100 cm water column (cm H₂O)) on the upper airways.

The active ingredients listed in Table 1 as Example 1, Example 3 and Example 4 were dissolved in the various formulations listed in Table 2 below and administered in an amount of 0 μg, 3 μg, 10 μg, 30 μg or 100 μg per pig. The active ingredient formulation or the pure vehicle was each administered with a pipette at a volume of 400 μl in each nostril.

TABLE 2
Compositions of the formulations for nasal administration in which
the compound listed in Table 1 as Example 3 was administered:

| | | | Glycerol | | | |
|---|---|---|---|---|---|---|
| | Phosphate | Polysor- | 85% | | | |
| | buffer | bate | (absolute | | Propylene | Na- |
| Formu- | pH 7 | 80 | glycerol) | PEG400 | glycol | CMC |
| lation | [% w/v] | [% w/v] | [% w/v] | [% w/v] | [% w/v] | [% w/v] |
| 1 | 90 | 10 | | | | |
| 2 | | | | 100 | | |
| 3 | 70 | 10 | | 20 | | |
| 4 | 85 | 10 | | 5 | | |
| 5 | 87.5 | 10 | | 2.5 | | |
| 6 | 67.5 | 10 | 2.5 | 20 | | |
| | | | (2.125) | | | |
| 7 | 70 | 10 | | | 20 | |
| 8 | 68.75 | 10 | | 20 | | 1.25 |
| 9 | 88.75 | 10 | | | | 1.25 |

The formulations of Table 2 optionally additionally comprise butylhydroxyanisole at a concentration of 0.02% w/v.

The phosphate buffer pH 7, 0.063 M was prepared according to the European Pharmacopoeia 8.7: 5.18 g of anhydrous disodium hydrogenphosphate and 3.65 g of sodium dihydrogenphosphate monohydrate were dissolved in 950 mL of water, the pH was adjusted with phosphoric acid and the solution made up to 1000 mL with water. Alternatively, the phosphate buffer was prepared using disodium hydrogenphosphate dihydrate and sodium dihydrogenphosphate dihydrate in place of the anhydrous disodium hydrogenphosphate and the sodium dihydrogenphosphate monohydrate. For this purpose, 6.49 g of disodium hydrogenphosphate dihydrate and 4.13 g of sodium dihydrogenphosphate dihydrate were dissolved in 950 mL of water, the pH was adjusted with phosphoric acid and the solution made up to 1000 mL with water.

The duration of action in this pig model is defined as the time [min] in which a collapse of the upper airways was not observed in any animal, as a mean value of the specified number of animals. A duration of action specified as “>” X min signifies that the experiment was terminated at X min and up to this point a collapse of the upper airways was still not observed in any animal

TABLE 3
Duration of action of Example 3/Table 1 in phosphate buffer pH 7/
polysorbate 80 with PEG400 (Formulation 3) or with 85% glycerol
and PEG400 (Formulation 6) in comparison to the duration of
action of Example 3/Table 1 in phosphate buffer pH 7/polysorbate
80 (Formulation 1)

| | | Glycerol 85% | Example | |
|---|---|---|---|---|
| Formulation | | (absolute | 3 from | |
| according to | PEG400 | glycerol) | Tab. 1 | Duration of action |
| Table 2 | [% w/v] | [% w/v] | [μg] | [min], mean value |
| 1 | 0 | 0 | 3 | 150 |
| 1 | 0 | 0 | 30 | 180* |
| 3 | 20 | 0 | 0 | 0 |
| 3 | 20 | 0 | 3 | 180 |
| 3 | 20 | 0 | 30 | 300 |
| 6 | 20 | 2.5 (2.125) | 3 | 240 |

*In experiments in which the nasal passages of the pigs were blocked by mucous, which were suggested by very noisy curves of the tracheal pressure and air flow, the mean value of the duration of action of 30 μg of Example 3 in Formulation 1 was 120 min.

TABLE 4
Duration of action of Example 3/Table 1 in phosphate buffer
pH 7/polysorbate 80/PEG, comparison of various PEG concentrations

| Formulation | | Example 3 | Duration of action |
|---|---|---|---|
| according to | PEG400 | from Table 1 | [min] |
| Table 2 | [% w/v] | [μg] | mean value |
| 2 | 100 | 3 | >240 |
| 3 | 20 | 3 | 180 |
| 4 | 5 | 3 | 180 |
| 5 | 2.5 | 3 | 150 |
| 1 | 0 | 3 | 150 |

TABLE 5
Duration of action of Example 3/Table 1 in phosphate buffer
pH 7/polysorbate 80 (90/10) + Na-CMC, Comparison with the
duration of action of Example 3/Table 1 in phosphate buffer pH 7/
polysorbate 80/PEG400 (70/10/20)

| Formulation | | Example 3 | Duration of |
|---|---|---|---|
| according to | Na-CMC/PEG400 | from Table 1 | action [min] |
| Table 2 | [% w/v] | [μg] | mean value |
| 9 | 1.25 Na-CMC | 3 | 120 |
| 3 | 20 PEG | 3 | 180 |

TABLE 6
Duration of action of Example 3/Table 1 in phosphate buffer pH 7/
polysorbate 80 with PEG (70/10/0) or propylene glycol (70/10/20)

| Formulation | PEG400/propylene | | Duration of |
|---|---|---|---|
| according to | glycol | Example 3 | action [min] |
| Table 2 | [% w/v] | [μg] | Mean value |
| 3 | 20 PEG | 30 | 300 |
| 7 | 20 propylene glycol | 30 | 180 |

TABLE 7
Duration of action of Example 1/Table 1 in phosphate buffer
pH 7/polysorbate 80/PEG400 (Formulation 3) in comparison
to the duration of action of Example 1/Table 1 in phosphate
buffer pH 7/polysorbate 80 (Formulation 1) at a reduced
pressure of −100 mbar and −50 mbar

| Formulation | Reduced | | Example 1 | Duration of |
|---|---|---|---|---|
| according to | pressure | PEG400 | from Tab. 1 | action[min], |
| Table 2 | [mbar] | [% w/v] | [μg] | mean value |
| 1 | −50 | 0 | 10 | 150 |
| 1 | −100 | 0 | 10 | 120 |
| 3 | −50 | 20 | 10 | >210 |
| 3 | −100 | 20 | 10 | 180 |

TABLE 8
Duration of action of Example 4/Table 1 in phosphate buffer
pH 7/polysorbate 80/PEG400 (Formulation 3) in comparison to
the duration of action of Example 4/Table 1 in phosphate buffer
pH 7/polysorbate 80 (Formulation 1) at a reduced pressure
of −100 mbar and −50 mbar

| Formulation | Reduced | | Example 4 | Duration of |
|---|---|---|---|---|
| according to | pressure | PEG400 | from Tab. 1 | action [min], |
| Table 2 | [mbar] | [% w/v] | [μg] | mean value |
| 1 | −50 | 0 | 100 | 180 |
| 1 | −100 | 0 | 100 | 180 |
| 3 | −50 | 20 | 100 | >210 |
| 3 | −100 | 20 | 100 | 180 |

1. A stable pharmaceutical formulation for nasal or pharyngeal administration comprising:
a therapeutically effective amount of at least one inhibitor of the TASK-1 and/or TASK-3 channel or a hydrate, solvate, polymorph or metabolite thereof, or a pharmaceutically acceptable salt thereof, in
>2.5% to 100% w/v polyethylene glycol,
wherein the formulation has a pH of 4 to 8.

2. The stable pharmaceutical formulation for nasal or pharyngeal administration according to claim 1, further comprising at least one auxiliary, wherein the at least one auxiliary is selected from the group consisting of at least one pH regulator, at least one solubilizer, at least one antioxidant, at least one stabilizer, at least one thickener, at least one preservative, at least one substance for adjusting tonicity, at least one aroma, at least one fragrance and at least one dye.

3. The stable pharmaceutical formulation for nasal or pharyngeal administration according to claim 2, wherein the at least one auxiliary comprises at least one pH regulator, and wherein the at least one pH regulator is selected from the group consisting of citric acid and salts thereof, acetic acid and salts thereof, phosphoric acid and salts thereof, hydrochloric acid, carboxylic acids, dicarboxylic acids, amino acids, oxocarboxylic acids, polycarboxylic acids, sodium hydroxide, potassium hydroxide, sodium carbonate and sodium hydrogencarbonate.

4. The stable pharmaceutical formulation for nasal or pharyngeal administration according to claim 2, wherein the at least one auxiliary comprises at least one solubilizer, and wherein the at least one solubilizer is selected from the group consisting of ethanol, polysorbate 20, polyoxyethylene (8) stearate and polysorbate 80.

5. The stable pharmaceutical formulation for nasal or pharyngeal administration according to claim 2, wherein the at least one auxiliary comprises at least one antioxidant, wherein the at least one antioxidant is selected from the group consisting of citric acid, butylhydroxyanisole, butylhydroxytoluene, EDTA and purging with nitrogen.

6. The stable pharmaceutical formulation for nasal or pharyngeal administration according to claim 2, wherein the at least one auxiliary comprises at least one preservative, and wherein the at least one preservative is selected from the group consisting of C₈-C₁₈ alkonium chloride, methylparaben, propylparaben, sorbic acid, chlorobutanol and benzalkonium chloride.

7. The stable pharmaceutical formulation for nasal or pharyngeal administration according to claim 1, wherein the formulation comprises 5 to 100% w/v polyethylene glycol 400, 0 to 10% w/v of a solubilizer, and 0 to 95% w/v of a pH regulator.

8. The stable pharmaceutical formulation for nasal or pharyngeal administration according to claim 1, wherein the at least one inhibitor of the TASK-1 and/or TASK-3 channel is selected from a compound of formula (I), 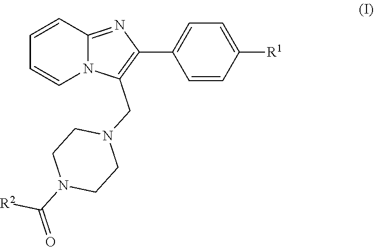
wherein
R¹ is halogen, cyano, (C₁-C₄)-alkyl, cyclopropyl or cyclobutyl;
R² is (C₄-C₆)-cycloalkyl, wherein a ring CH₂ group may be replaced by —O—,
or
R² is represents a phenyl group of formula (a) or a pyridyl group of formula (b) 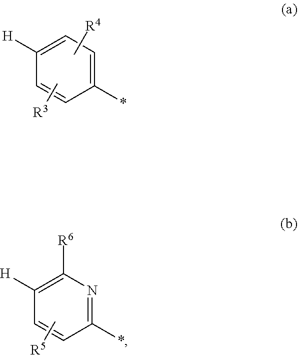
wherein * marks the bond to the adjacent carbonyl group; and
R³ is fluorine, chlorine, bromine, cyano, (C₁-C₃)-alkyl or (C₁-C₃)-alkoxy, wherein (C₁-C₃)-alkyl and (C₁-C₃)-alkoxy may be up to trisubstituted by fluorine;
R⁴ is hydrogen, fluorine, chlorine, bromine or methyl;
R⁵ is hydrogen, fluorine, chlorine, bromine or methyl;
and
R⁶ is hydrogen, (C₁-C₃)-alkoxy, cyclobutyloxy, oxetan-3-yloxy, tetrahydrofuran-3-yloxy or tetrahydro-2H-pyran-4-yloxy,
wherein (C₁-C₃)-alkoxy may be up to trisubstituted by fluorine,
or a hydrate, solvate, polymorph or metabolite thereof, or a pharmaceutically acceptable salt thereof.

9. The pharmaceutical formulation for nasal or pharyngeal administration according to claim 8, wherein the at least one inhibitor of the TASK-1 and/or TASK-3 channel is selected from the group consisting of:
(4-{[2-(4-chlorophenyl)imidazo[1,2-a]pyridin-3-yl]methyl}piperazin-1-yl)(6-methoxypyridin-2-yl)methanone,
(4-{[2-(4-bromophenyl)imidazo[1,2-a]pyridin-3-yl]methyl}piperazin-1-yl)(2-fluorophenyl)methanone,
(4-{[2-(4-Bromophenyl)imidazo[1,2-a]pyridin-3-yl]methyl}piperazin-1-yl)(cyclopentyl)methanone, and
(4-{[2-(4-chlorophenyl)imidazo[1,2-a]pyridin-3-yl]methyl}piperazin-1-yl)(cyclopentyl)methanone,
or a hydrate, solvate, polymorph or metabolite thereof, or a pharmaceutically acceptable salt thereof.

10. The stable pharmaceutical formulation according to claim 9, wherein the at least one inhibitor of the TASK-1 and/or TASK-3 channel is (4-{[2-(4-chlorophenyl)imidazo[1,2-a]pyridin-3-yl]methyl}piperazin-1-yl)(6-methoxypyridin-2-yl)methanone or a hydrate, solvate, polymorph or metabolite thereof, or a pharmaceutically acceptable salt thereof.

11. (canceled)

12. A method for treatment or prevention of respiratory disorders, sleep-related respiratory disorders, obstructive sleep apnoeas, central sleep apnoeas, snoring, cardiac arrhythmias, arrhythmias, neurodegenerative disorders, neuroinflammatory disorders or neuroimmunological disorders, comprising administering to a patient in need thereof a therapeutically effective amount of a stable pharmaceutical formulation according to claim 1.

13. A method for treatment or prevention of respiratory disorders, sleep-related respiratory disorders, obstructive sleep apnoeas, central sleep apnoeas, snoring, cardiac arrhythmias, arrhythmias, neurodegenerative disorders, neuroinflammatory disorders or neuroimmunological disorders, comprising administering to a patient in need thereof a therapeutically effective amount of a stable pharmaceutical formulation according to claim 1, wherein the formulation is by nasal sprays, nasal drops, nasal solutions, powder inhalers, nebulizers, metered dose aerosols or semisolid gels.

14. A method for treatment or prevention of respiratory disorders, sleep-related respiratory disorders, obstructive sleep apnoeas, central sleep apnoeas, snoring, cardiac arrhythmias, arrhythmias, neurodegenerative disorders, neuroinflammatory disorders or neuroimmunological disorders, comprising administering to a patient in need thereof a therapeutically effective amount of a stable pharmaceutical formulation according to claim 1, wherein the duration of action is at least 4 hours.

15. A method for treatment or prevention of obstructive sleep apnoeas or snoring, comprising administering to a patient in need thereof a stable pharmaceutical formulation according to claim 8, wherein the stable pharmaceutical formulation comprises:
a therapeutically effective amount of the inhibitor of the TASK-1 and/or TASK-3 channel 4-{[2-(4-chlorophenyl)imidazo[1,2-a]pyridin-3-yl]methyl}piperazin-1-yl)(6-methoxypyridin-2-yl)methanone or a hydrate, solvate, polymorph or metabolite thereof or a pharmaceutically acceptable salt thereof, in
20% to 100% w/v PEG400, and
0 to 10% w/v polysorbate 80 and 0 to 80% w/v of a phosphate buffer having a pH of 7,
wherein the duration of action of the stable pharmaceutical formulation after nasal or pharyngeal administration is at least 5 hours.

16. The stable pharmaceutical formulation according to claim 7, wherein the stable pharmaceutical formulation comprises at least one further auxiliary.

17. The method according to claim 15, wherein the stable pharmaceutical formulation comprises at least one further auxiliary.
